SEQUENCE LISTING (4 sequences)

SEQ ID NO: 1 - protein, 103 aa, Klebsiella pneumoniae
MELRMREISQKDLNLAFGAGETDPNTQLLNDLGNNMAWGAALGAPGGLGSAALGAAGGALQTVGQGLIDHGPVNVPIPVLIGPSWNGSGSGYNSATSSSGSGS

SEQ ID NO: 2 - protein, 84 aa, Klebsiella pneumoniae
GETDPNTQLLNDLGNNMAWGAALGAPGGLGSAALGAAGGALQTVGQGLIDHGPVNVPIPVLIGPSWNGSGSGYNSATSSSGSGS

SEQ ID NO: 3 - protein, 90 aa, Escherichia coli
MYMRELDREELNCVGGAGDPLADPNSQIVRQIMSNAAWGPPLVPERFRGMAVGAAGGVTQTVLQGAAAHMPVNVPIPKVPMGPSWNGSKG

SEQ ID NO: 4 - protein, 74 aa, Escherichia coli
AGDPLADPNSQIVRQIMSNAAWGPPLVPERFRGMAVGAAGGVTQTVLQGAAAHMPVNVPIPKVPMGPSWNGSKG

(19) United States
(12) Patent Application Publication
(10) Pub. No.: US 2005/0159349 A1
(43) Pub. Date: Jul. 21, 2005

(54) Apoptogenic-bacteriocins combining broad spectrum antibiotic and selective anti-tumoral activities, and compositions and uses thereof

(76) Inventors: Claudio Soto, Friendswood, TX (US); Rosalba Lagos, Santiago (CL)

Correspondence Address: KLAUBER & JACKSON, 411 HACKENSACK AVENUE, HACKENSACK, NJ, 07601, US

(21) Appl. No.: 10/506,857

(22) PCT Filed: Mar. 5, 2003
(86) PCT No.: PCT/IB03/00821

(30) Foreign Application Priority Data
Mar. 5, 2002 (US) 60361804

(51) Int. Cl. A61K 38/16; C07K 14/195

(52) U.S. Cl. 514/12; 530/350

(57) ABSTRACT

The present invention relates generally to bacterial proteins, particularly to apoptogenic-bacteriocins, which are capable of killing other bacteria and of also inducing apoptosis in certain eukaryotic cells. The apoptogenic-bacteriocin of the present invention produces apoptosis in tumor-derived cells or cancer cells, while being not cytotoxic to normal cells and not inducing any evident toxicityin vivo in normal animals. Uses of the apoptogenic-bacteriocin and methods of treating cancer, tumors and other diseases of the aberrant cell growth are provided.

### FIELD OF THE INVENTION

The present invention relates generally to bacterial proteins, particularly to apoptogenic-bacteriocins, which are capable of killing other bacteria and of also inducing apoptosis in certain eukaryotic cells. The apoptogenic-bacteriocin of the present invention produces apoptosis in tumor-derived cells or cancer cells, while being not cytotoxic to normal cells and not inducing any evident toxicity in vivo in normal animals. Uses of the apoptogenic-bacteriocin and methods of treating cancer, tumors and other diseases of aberrant cell growth are provided.

### BACKGROUND OF THE INVENTION

Bacteriocins are antimicrobial proteins produced by bacteria that are active against closely related species. They are produced by almost all major lineages of Eubacteria and in Archaebacteria (Riley and Gordon, 1999) and secreted across the cell envelope to the extracellular medium, where they recognize specific receptors located on the surface of sensitive cells and are subsequently translocated to their specific intracellular targets (Kolter and Moreno, 1992). It has been proposed that the primary role of bacteriocins is to mediate population dynamics within species (Riley and Gordon, 1999). Bacteriocins can induce toxicity by a variety of mechanisms, most notably the formation of membrane pores, a mechanism which has brought increasing attention to bacteriocins for their potential use as preservatives in the food industry, and as antibiotics for clinical usage (Jack et al., 1995; Riley, 1998; Baba and Schneewind, 1998; Ennahar et al., 2000).

Bacteria also produce other toxic proteins, released by pathogens into the host's body, that are important virulence factors causing disease symptoms (tetanus, diphtheria, cholera, etc.). These toxic agents are commonly called bacterial toxins, and may attack a variety of different cell types (cytotoxins), or specific cell types (neurotoxins, hemolysins, etc). Among these bacterial toxins some can form membrane pores, and kill eukaryotic cells by disrupting the membrane permeability barrier. Lately, increasing interest has developed in the study of some toxins for their potential therapeutic use as antitumoral drugs. Thus, cytotoxic studies of toxins such as verotoxin 1 (Farkas-Himsley et al., 1995), and a fusion protein derivative of a Pseudomonas exotoxin, using IL-4 to target cancer cells (Leland et al., 2000), have demonstrated that these toxins display antitumor activity in vitro and in vivo in ovarian and breast cancer, respectively. Another aspect of the study of bacterial toxins that has aroused considerable interest, is the role of toxin-producing pathogens in promoting apoptosis (Weinrauch and Zychlinsky, 1999). In contrast to necrosis, apoptosis eliminates cells without inducing an inflammatory response, i.e. in an immunologically silent manner. Thus, induction of apoptosis by pathogens may be a way to subvert the normal host defense responses. Among the pore-forming toxins that induce apotosis is the major cytolysin from Staphylococcus aureus, α-toxin α-hemolysin) (Dinges et al., 2000). α-Toxin forms pores in T-lymphocytes, inducing an uncontrolled sodium influx, which in turn provokes the release of Ca²⁺ from intracellular stores (Jonas et al., 1994). Aerolysin, another channel-forming toxin produced by Aeromonas hydrophyla, triggers apoptosis of T lymphomas probably by an increase in intracellular Ca²⁺ (Nelson et al., 1999). Neisseria gonorrhoeae induces apoptosis in epithelial and phagocytic cell lines by translocation of a porin (PorB) from the outer membrane of the bacteria into the target cells (Müller et al., 1999). This causes a rapid Ca²⁺ influx followed by the activation of proteases belonging to the caspase family and to the Ca²⁺ dependent calpain family (Miller et al., 1999). PorB is transported to the mitochondria, where it exerts its action by promoting the release of cytochrome c (Müller et al., 2000).

Apotosis is a genetically determined form of cell death that plays a central role during development and homeostasis of multicellular organisms (Vaux and Korsmeyer, 1999; Jacobson et al., 1997). On the other hand, necrotic cell death is usually the consequence of physical injury and does not involve the active participation of the cell. Apoptosis can be distinguished from necrosis on the bases of several morphological as well as biochemical parameters, such as nuclear condensation, loss of cell volume (shrinkage) and DNA fragmentation (Jacobson et al., 1997). A critical stage of apoptosis is phosphatidylserine exposure to the outer face of plasma membrane that results in the recognition and uptake of these dying cells by phagocytes (Savill and Fadok, 2000). This kind of cell death avoids spillage of intracellular contents in contrast to necrotic cell death, typified by cell and organelle swelling and membrane disruption, resulting in an inflammatory response (Savill and Fadok, 2000). The central executioners of apoptosis are a set of cysteine proteases that are part of a large protein family known as caspases (Hengartner, 2000). Based on structural similarities and substrate preference, caspases have been divided into many subfamilies. Activation of caspases that led to apoptosis may be initiated by cell surface receptors or by mitochondrial stress, and each of these pathways are initially executed by different caspases. On the other hand, inhibition of caspase activity prevents apoptosis (Hengartner, 2000).

Some bacterial toxins share common features with bacteriocins, such as insertion into or transport across the plasma membrane, recognition of the target cell's membrane, and pore formation. In spite of these similarities the action of bacterial toxins and bacteriocins are restricted to eukaryotic cells and bacteria, respectively, probably due to specific interactions between the receptor and the toxin.

Therefore, it is evident that there still exists a need in the art for a protein or composition that acts on bacterial and eukaryotic cells, and particularly that can exert directed effects or cell killing on specific eukaryotic cells, particularly tumor or cancer cells or other cells demonstrating aberrant cell growth.

The citation of references herein shall not be construed as an admission that such is prior art to the present invention.

### SUMMARY OF THE INVENTION

In its broadest aspect, the present invention extends to bacterial apoptogenic-bacteriocins and to their capability to kill bacterial cells and induce apoptosis in particular eukaryotic cells. In particular, bacterial proteins are provided which induce apoptosis in eukaryotic tumor cells, cancer cells, or cells undergoing aberrant growth and which are not toxic to normal eukaryotic cells.

In a particular aspect of the invention, an apoptogenic-bacteriocin protein is provided, including in a preferred embodiment a pore-forming or channel forming bacterial protein of low molecular weight, particularly of less than 10,000 MW. In a preferred embodiment of the invention, a low molecular weight pore-forming or channel forming apoptogenic-bacteriocin is provided, from gram positive or gram negative bacteria, having a molecular weight of about 2,500 to about 10,000.

In a preferred embodiment, the bacteriocin microcin E492 is provided for use in killing bacterial cells and in inducing apoptosis in eukaryotic tumor cells, cancer cells, or cells undergoing aberrant growth. In a particular embodiment, microcin E492 comprising the amino acid sequence set out in FIGS. 6 and 7 (SEQ ID NOS: 1 and 2) is provided. In a further particular embodiment, processed microcin E492 comprising the amino acid sequence of SEQ ID NO: 2 is provided.

In a further embodiment, the bacteriocin microcin 24 is provided for use in killing bacterial cells and in inducing apoptosis in eukaryotic tumor cells, cancer cells, or cells undergoing aberrant growth. In a particular embodiment, microcin 24 comprising the amino acid sequence set out in FIG. 8 (SEQ ID NO: 3) is provided. In a further particular embodiment, processed microcin 24 comprising the amino acid sequence of SEQ ID NO: 4 is provided.

In a still further embodiment, the present invention includes fusion proteins or derivatives of the apoptogenic-bacteriocin, wherein the apoptogenic-bacteriocin is covalently or non-covalently linked to an anti-cancer or anti-tumor compound or agent or to a compound, agent or polypeptide which targets or otherwise directs the apoptogenic-bacteriocin to tumor or cancer cells.

In a still further aspect, the present invention extends to a pharmaceutical composition of an apoptogenic-bacteriocin, comprising the apoptogenic-bacteriocin and a pharmaceutically acceptable carrier, vehicle or diluent.

In a particular aspect, a pharmaceutical composition is provided comprising microcin E492 and a pharmaceutically acceptable carrier, vehicle or diluent. In a further aspect, a pharmaceutical composition is provided comprising the polypeptide of SEQ ID NO: 1 or SEQ ID NO: 2 and a pharmaceutically acceptable carrier, vehicle or diluent.

In a particular aspect, a pharmaceutical composition is provided comprising microcin 24 and a pharmaceutically acceptable carrier, vehicle or diluent. In a further aspect, a pharmaceutical composition is provided comprising the polypeptide of SEQ ID NO: 4 and a pharmaceutically acceptable carrier, vehicle or diluent.

The present invention also relates to a recombinant DNA molecule or cloned gene, or a degenerate variant thereof, which encodes an apoptogenic-bacteriocin, preferably a nucleic acid molecule; in particular a recombinant DNA molecule or cloned gene that has a nucleotide sequence or is complementary to a DNA sequence capable of encoding the amino acid sequence set out in FIGS. 6, 7 or 8 or in SEQ ID NO: 1, SEQ ID NO: 2, SEQ ID NO:3 or SEQ ID NO:4, or an active portion or analog thereof.

The DNA sequences of the apoptogenic-bacteriocins of the present invention or portions thereof, particularly those encoding the polypeptides of any of SEQ ID NOS: 1, 2, 3, or 4, or a portion thereof, may be prepared as probes to screen for complementary or homologous sequences in other bacterial clinical isolates or other bacteria, including gram negative and gram positive bacteria. The present invention extends to probes so prepared that may be provided for screening nucleic acid libraries for the apoptogenic-bacteriocins. The present invention also includes the preparation of plasmids including such vectors, and the use of the DNA sequences to construct vectors expressing antisense RNA or ribozymes which would attack the mRNAs of any or all of the DNA sequences. Correspondingly, the preparation of antisense RNA and ribozymes are included herein.

In a further embodiment of the invention, the full DNA sequence of the recombinant DNA molecule or cloned gene so determined may be operatively linked to an expression control sequence which may be introduced into an appropriate host. The invention accordingly extends to unicellular hosts transformed with the cloned gene or recombinant DNA molecule comprising a DNA sequence encoding the present apoptogenic-bacteriocins(s), and more particularly, the DNA sequence capable of encoding the polypeptides of any of SEQ ID NOS: 1, 2, 3 or 4.

The present invention extends to the development of antibodies against the apoptogenic-bacteriocin(s) of the present invention, including in particular embodiments microcin E492 and microcin 24, including naturally raised and recombinantly prepared antibodies. Such antibodies include both polyclonal and monoclonal antibodies prepared by known genetic techniques, as well as bi-specific (chimeric) antibodies, humanized antibodies, synthetic antibodies, active fragments and antibodies including other functionalities suiting them for additional diagnostic or therapeutic use. Such antibodies are useful in methods to determine the quantity, amount or activity of the apoptogenic-bacteriocin and/or to modulate the activity of the apoptogenic-bacteriocin, particularly in the case of blocking or neutralizing antibodies.

In a further embodiment, the present invention relates to therapeutic methods based upon the activity of the apoptogenic-bacteriocin(s), its (or their) subunits, or active fragments thereof, analogs thereof, or upon agents or other drugs determined to possess the same activity. A first therapeutic method is associated with the apoptosis of tumor or cancer cells using the apoptogenic-bacteriocin(s), either individually or in mixture with each other in an amount effective to prevent, slow or inhibit the development of tumors or cancer in the host, particularly in a mammal, most particularly in a human. A further method is provided for apoptosis of tumor or cancer cells using the apoptogenic-bacteriocin(s), either individually or in mixture with each other, in an amount effective to reduce the size or growth of tumors or cancer in the host. In a further aspect, methods are provided for apoptosis of tumor or cancer cells using the apoptogenic-bacteriocin(s) in combination with an anti-tumor or anti-cancer agent or compound.

In a still further aspect, the present invention provides methods of reducing or blocking eukaryotic cell growth by administration of an apoptogenic-bacteriocin, particularly by administration of microcin E492 and/or microcin 24. In a further aspect, a method is provided for apoptosis of cells undergoing aberrant cell growth. In a particular aspect, a method is provided for treatment or prevention of conditions associated with aberrant cell growth by administration of an apoptogenic-bacteriocin, particularly by administration of microcin E492 and/or microcin 24. In particular embodiments, methods are provided for treatment or prevention of conditions associated with aberrant cell growth, including but not limited to benign and malignant tumors, cancers, hyperplasias, tissue hypertrophies, psoriasis and polyps, by administration of an apoptogenic-bacteriocin, particularly by administration of microin E492 and/or microcin 24, in a particular embodiment by administration of a pharmaceutical composition comprising a polypeptide comprising the amino acid sequence of SEQ ID NO: 1, 2, 3 or 4.

The methods of the present invention include a method for the treatment or prevention of cancer by the administration of pharmaceutical compositions comprising the apoptogenic-bacteriocin(s) of the present invention. In a particular embodiment, a method for treatment or prevention of cancer is provided comprising the administration of a pharmaceutical composition comprising microcin E492 and/or microcin 24. In a further embodiment, a method for treatment or prevention of cancer is provided comprising the administration of a pharmaceutical composition comprising a polypeptide comprising the amino acid sequence set out in any of SEQ ID NOS: 1, 2, 3 or 4. In a still further embodiment, a method for treatment or prevention of cancer is provided comprising the administration of a pharmaceutical composition comprising one or more of microcin E492 and microcin 24, in combination with an anti-tumor or anti-cancer agent or compound.

Other objects and advantages will become apparent to those skilled in the art from a review of the following description which proceeds with reference to the following illustrative drawings.

### BRIEF DESCRIPTION OF THE DRAWINGS

FIG. 1A-1F. Microcin E492 induces cell death with DNA fragmentation in a dose-dependent manner. HeLa cells (0.4×10⁶ cells in one mL) were incubated during 24 h under different conditions and analyzed by the propidium iodide (PI) exclusion method. (A) Untreated control. (B) Cells incubated with 256 AU of microcin E492. (C) Cells incubated with 1024 AU of microcin E492. (D) As negative control cells were incubated with 10 μg of a preparation from the mutant strain E. coli VCS257np108 which does not produce microcin E492. The inset shows a PAGE of microcin E492 (labeled with fluorescamine) purified from the microcin producing strain E. coli VCS257pJEM15 (lane 1) and of a purified fraction from the non-producing strain E. coli VCS257 np108 (lane 2). The arrow indicates the microcin protein band, and lane 3 corresponds to the molecular weight markers. (E) As a positive control cells were treated with 1 ng/mL TNF-α and 5 μg/mL cycloheximide. (F) Total DNA content (without degradation) was measured by permeabilization of non-treated cells with 95% methanol and staining with PI. 10,000 cells per sample were analyzed by flow cytometry and cell counts were plotted against PI fluorescence. Dead cells permeable to PI are indicated in the diagram as M1 and M2 populations. M0 population represents viable cells, not permeable to PI. mcc, microcin E492.

FIGS. 2A and 2B. Microcin E492 induces chromatin condensation and DNA degradation. (A) HeLa cells (1 mL) were seeded onto glass coverslips and incubated with or without 512 AU of microcin E492 for 24 h. PI was added and nuclear morphology was analyzed by phase contrast microscopy (left) or confocal microscopy (right). Phase contrast and fluorescence pictures were taken on the same section. Pictures are the representative result of three different experiments. (B) HeLa cells (1 mL) were incubated with or without 250 AU of microcin E492 for 18 h. Apoptotic cells with fragmented DNA were detected in situ by the TUNEL assay. Left, non-treated cells; center, cells incubated with DNase I as a positive control; right, cells treated with microcin 1492. Apoptotic nuclei are shown in green (dUTP-FITC incorporation) and total nuclei are shown in red (PI staining).

FIG. 3A-3E. Microcin E492 induces caspase activation and mitochondrial dysfunction. HeLa cells (0.2×10⁶ cells in one mL) were treated with 500 AU/mL of microcin E492 for 10 h, examined by flow cytometry, and the light scattering properties (A) were assessed on a forward scatter versus side scatter dot plot, while the mitochondrial membrane potential (B) of these samples was evaluated staining the cells with DiOC₆(3). The percentage of cells with decreased cell volume or mitochondrial membrane potential are indicated. NT corresponds to the untreated control. (C) HeLa cells (15×10⁶ cells in one mL) were incubated with 700 AU of microcin E492 for 7 h, the cell extracts were separated into cytoplasmic (SN) and nuclear-mitochondrial (P) components and analyzed for the presence of cytochrome c by immunoblotting. (D) HeLa cells treated with different amounts of microcin E492 for 12 h were disrupted and incubated with 50 μM of caspase-1 substrate Ac-YVAD-pma or caspase-3 substrate Ac-DEVD-pma for caspase activity quantification. The results presented are the average of two independent experiments. (E) HeLa cells were incubated with or without 100 μM of Ac-DEVD-cho (DEVD), Ac-YVAD-cho (YVAD) or z-VAD-fink (zVAD) for 2.5 h before addition of 700 AU of microcin E492. After 24 h, cells were collected and stained with PI for cell viability analysis by FACS. The results correspond to the mean and standard deviations of three independent assays.

FIGS. 4A and 4B. Microcin E492 dose effect on the induction of necrosis and apoptosis in HeLa cells. One L of cells were incubated with different doses of microcin E492 for 12 h. (A) Phosphatidylserine exposure on the external plasma membrane leaflet was assessed by annexin V-FITC binding, and loss of membrane integrity by PI uptake. 20,000 cells per sample were analyzed by flow cytometry. As positive control, HeLa cells were incubated with 1 ng/mL TNF-α and 5 μg/mL cycloheximide. The bars are the average of 3 determinations. 100% of cells corresponds to the addition of viable, apoptotic and necrotic cells. (B) Determination of LDH release to the culture medium was assessed measuring LDH activity from the supernatants. One B-B(LDH) unit is defined as the amount of LDH that reduces 4.8×10⁻⁴ μmol of piruvate per min at 37° C.

FIG. 5A-5E. Microcin E492 induces an increase of intracellular Ca²⁺ levels as the result of Ca²⁺ release from intracellular stores. HeLa cells (A, B, D, E) were loaded with the Ca²⁺ dye Fluo-3 (10 μg/mL final concentration). Changes in fluorescence intensity were measured by confocal microscopy. Relative fluorescence intensity (variations of fluorescence intensity normalized by the basal fluorescence) was plotted against time, and represents the average of twenty individual cells. (A) Addition of microcin E492 (arrow) to a final concentration of 512 AU/mL. The inset shows the variation of intracellular Ca²⁺ levels after 30 min of treatment with different doses of microcin E492. (B) Treatment of HeLa cells with a preparation from the supernatant of a non-producing strain (negative control, first arrow), followed by the addition of 512 AU/mL of microcin E492 (positive control, second arrow). (C) 512 AU/mL of microcin E492 were added to AMG-3 cells (first arrow). As control of Ca²⁺ loading, cells were treated with 2 μg/ml of ionomycin (second arrow). (D) HeLa cells were treated with 250 AU/mL of microcin in the presence of 5 mM EGTA (first arrow). Then, the solution was replaced with fresh KRH-glc (which contains 1.3 mM CaCl₂) plus 256 AU/mL of microcin E492 (second arrow). After 10 min of incubation this solution was replaced by KRH-glc containing 512 AU/mL of microcin E492 (third arrow). (E) High doses of microcin E492 induce morphological features of necrotic cell death. HeLa cells loaded with Fluo-3 were incubated with 2048 AU/mL of microcin E492. Top, untreated cells; center, after 20 min; and bottom, after 25 min of incubation with microcin E492. Arrows show the appearance of blebs at the cell surface.

FIG. 6 depicts the amino acid sequence of the unprocessed 103 amino acid microcin E492 (SEQ ID NO:1).

FIG. 7 depicts the amino acid sequence of the processed and active 84 amino acid microcin E492 (SEQ ID NO:2).

FIG. 8 depicts the amino acid sequence of unprocessed microcin 24 (SEQ ID NO:3). The double glycine motif for processing of microcin 24 is underlined and the arrow after the second glycine depicts predicted processing to a processed microcin 24 of 74 amino acids (SEQ ID NO:4).

### DETAILED DESCRIPTION

The present invention provides a bacterial protein, an apoptogenic-bacteriocin, that has the ability to both kill other bacteria and induce apoptosis of some specific types of eukaryotic cells. The apoptogenic-bacteriocin of the present invention has been found to produce apoptosis in tumor-derived cells, while it was not cytotoxic to normal cells nor induced any evident toxicity when injected intreperitonealy (i.p.) in vivo in normal animals.

An example of this apoptogenic-bacteriocin protein provided herein is the protein named microcin E492. Microcin E492 is a low-molecular-weight (7887 Da sequence deduced mass) channel-forming bacteriocin produced by gram negative Klebsiella pneumoniae RYC492 (Lagos et al., 1993, 1999). Microcin E492 is the first member of a new class of proteins having at the same time bacteriocin and bacterial toxin activities. This class of proteins will be called herein “apoptogenic-bacteriocins” and will be defined as having a potent activity to kill a broad range of prokaryotic cells and a selective apoptotic activity on eukaryotic cells.

The exemplification provided herein of apoptogenic-bacteriocin(s) using microcin E492 is provided merely by way of non-limiting example and is indicative of the character, function and activity of apoptogenic-bacteriocins, and is not intended to limit or otherwise restrict the invention or the breadth of the apoptogenic-bacteriocins as a class of proteins.

The only bacteriocin reported to be toxic for eukaryotic as well as prokaryotic cells is a cytolysin from Enterococcus faecalis. This toxin is related to lantibiotics, which belong to an antibiotic family secreted by gram-positive bacteria and which contains lanthionine derived from posttranslational modifications (Booth et. al, 1996).

Microcin E492 has a broad target range among prokaryotic cells, being active on strains of Escherichia coli, Klebsiella, Salmonella, Citrobacter, Enterobacter and Erwinia (de Lorenzo, 1984). The genetic determinants involved in the production of active microcin and its immunity protein have been cloned and expressed in E. coli (Wilkens et al., 1997), and comprise a 13 kb DNA fragment encoding at least 10 genes. Most of these genes are necessary for the synthesis and export of active microcin (Lagos et al., 2001). Analysis of the amino acid sequence of microcin E492 indicates that this apoptogenic-bacteriocin does not share structural motif similarity with any other known or studied protein, and thus, it seems that this microcin belongs to a novel class of pore-forming bacteriocins not related to colicin-like channel forming bacteriocin from gram negative bacteria or to channel-forming bacteriocins from gram positive bacteria (e.g., lantibiotics) (Lagos et al., 1999).

Analysis of the sequence of microcin B492 by FASTA and TBLASTN analysis reveals similarity to only one Genbank database entry. The gene mceA is homologous to the gene mtfS, the structural gene of microcin 24, isolated from an E. coli uropathogenic strain (O'Brien, G. J. and Mahanty, H. K. (1996) Genbank accession no. U47048). The microcin 24 and microcin E492 proteins are similar in size and demonstrate sequence identity of 52% and sequence similarity of 59%.

As demonstrated herein, microcin E492 promotes morphological changes typical of apoptosis such as chromatin condensation, cell shrinkage, DNA fragmentation and phosphatidylserine flipping to the outer monolayer of plasma membrane in certain eukaryotic cells or cell lines. Similarly to other eukaryotic toxins, microcin E492 induces a fast increase in cytosolic Ca²⁺, probably due to a release from intracellular stores. Interestingly, among the cells studied, microcin E492 produced apoptosis only on some tumor-derived cells, while it was not cytotoxic to normal cells nor induced any evident toxicity when injected intraperitoneally (i.p.) in vivo in normal animals. Therefore, apoptogenic-bacteriocins, and in a particular embodiment microcin E492, may have important applications in the treatment of cancer, by inducing the specific death of tumoral cells. At the same time, microcin E492 could have a beneficial antibiotic activity due to its ability to kill a broad range of bacterial strains.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

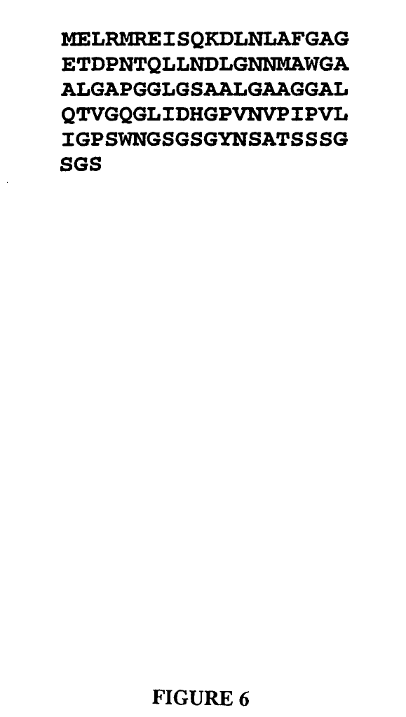

The terms “apoptogenic-bacteriocin”, “apoptogenic-bacteriocins” and any variants not specifically listed, may be used herein interchangeably, and as used throughout the present application and claims refer to proteinaceous material including single or multiple proteins, monomers or multimers, and extends to and includes those exemplary proteins having the amino acid sequence data described herein and presented in FIGS. 6, 7 and 8 (SEQ ID NOS: 1, 2, 3 and 4), and the profile of activities set forth herein and in the claims. The apoptogenic-bacteriocins described herein are low molecular weight bacterial proteins, having the ability to kill bacteria and induce apoptosis or cell death of eukaryotic cells, particularly including cancer or tumor cells or other cells undergoing aberrant cell growth. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits, or natural variants found in other isolates or bacterial strains. Also, the terms “microcin E492” and “microcin 24” are intended to include within their scope proteins specifically recited herein as well as all substantially homologous analogs, mutants and allelic variants.

The amino acid residues described herein are preferred to be in the “L” isomeric form. However, residues in the “D” isomeric form can be substituted for any L-amino acid residue, as long as the desired fuctional property of the apoptogenic-bacteriocin is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243: 3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences capable of encoding an apoptogenic-bacteriocin having the amino acid sequence set out in SEQ ID NO: 1, 2, 3 or 4, one such example being the DNA sequence set out in SEQ ID NO: 5, but which are degenerate to one another. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or |
| | AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA |
| | (opal) |

It should be understood that the codons specified above are for RNA sequences. The corresponding codons for DNA have a T substituted for U. Alterations or mutations can be made in the amino acid sequence of the apoptogenic-bacteriocin(s) of the present invention by directly changing the amino acid, for example by amino acid synthesis of an apoptogenic-bacteriocin peptide with altered sequence, or by changes in the DNA or nucleic acid sequence to encode an apoptogenic-bacteriocin peptide with altered sequence. Mutations can be made in the DNA sequences of the present invention such that a particular codon is changed to a codon which codes for a different amino acid. Such a mutation is generally made by making the fewest nucleotide changes possible. A substitution mutation of this sort can be made, to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative and non-conservative changes which do not significantly alter the activity or binding characteristics of the resulting protein.

The apoptogenic-bacteriocin(s) of the present invention further include mutants, analogs or derivatives including but not limited to, for example, mutations or alterations to the original amino acid sequence, fragments or deletions of the original apoptogenic-bacteriocin(s) including shortening the sequence to the minimally required active motif, modified peptides (for instance end-protected, alkylated at the alpha-carbon or amide nitrogen, cyclized peptides, retro-inverse peptides, peptides containing non-natural amino acids), psudopeptides, peptoids, azapeptides and peptide mimetics. These apoptogenic-bacteriocin mutants, analogs or derivatives should still retain some functional capacity in cell recognition and killing, and may also have increased potency, altered or enhanced stability and/or bioavailability, reduced protease degradation, reduced immunogenicity or antigenicity, or reduced toxicity, for example.

The following is one example of various groupings of amino acids:
Amino acids with nonpolar R groups: Alanine, Valine, Leucine, Isoleucine, Proline, Phenylalanine, Tryptophan, Methionine
Amino acids with uncharged polar R groups: Glycine, Serine, Threonine, Cysteine, Tyrosine, Asparagine, Glutamine
Amino acids with charged polar R groups (negatively charged at Ph 6.0): Aspartic acid, Glutamic acid
Basic amino acids (positively charged at pH 6.0): Lysine, Arginine, Histidine (at pH 6.0)
Another grouping may be those amino acids with phenyl groups: Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces—turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

In its broadest aspect, the present invention extends to bacterial apoptogenic-bacteriocins and to their capability to kill bacterial cells and induce apoptosis in particular eukaryotic cells. In particular, bacterial proteins are provided which induce apoptosis in eukaryotic tumor cells, cancer cells, or cells undergoing aberrant growth and which are not toxic to normal eukaryotic cells.

In a particular aspect of the invention, an apoptogenic-bacteriocin protein is provided, including in a preferred embodiment a pore-forming or channel forming bacterial protein of low molecular weight, particularly of less than 10,000 MW. In a preferred embodiment of the invention, a low molecular weight pore-forming or channel forming apoptogenic-bacteriocin is provided, from gram positive or gram negative bacteria, having a molecular weight of about 2,500 to about 10,000.

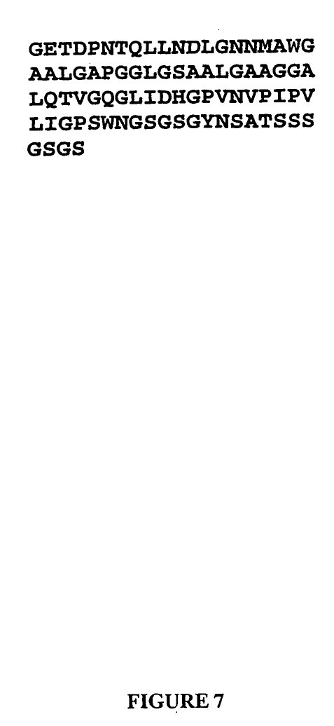

In a preferred embodiment, the bacteriocin microcin E492 is provided for use in killing bacterial cells and in inducing apoptosis in eukaryotic tumor cells, cancer cells, or cells undergoing aberrant growth. In a particular embodiment, microcin E492 comprising the amino acid sequence set out in FIGS. 6 and 7 (SEQ ID NOS: 1 and 2) is provided. In a further particular embodiment, processed microcin E492 comprising the amino acid sequence of SEQ ID NO: 2 is provided.

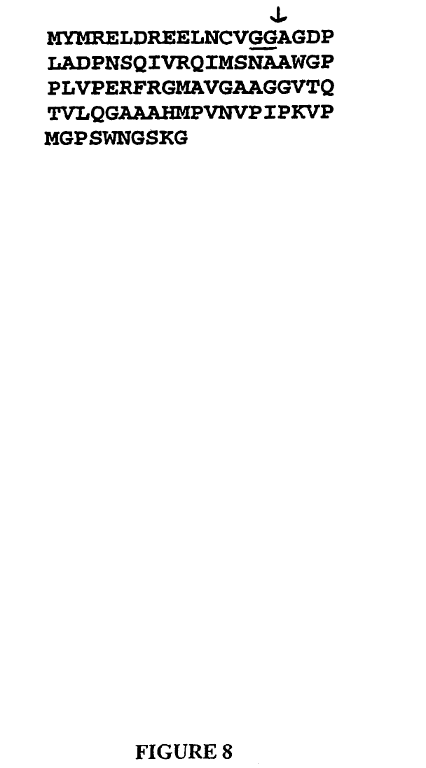

In a further embodiment, the bacteriocin microcin 24 is provided for use in killing bacterial cells and in inducing apoptosis in eukaryotic tumor cells, cancer cells, or cells undergoing aberrant growth. In a particular embodiment, microcin 24 comprising the amino acid sequence set out in FIG. 8 (SEQ ID NO: 3) is provided. In a further particular embodiment, processed microcin 24 comprising the amino acid sequence of SEQ ID NO: 4 is provided.

In a still further embodiment, the present invention includes fusion proteins or derivatives of the apoptogenic-bacteriocin, wherein the apoptogenic-bacteriocin is covalently or non-covalently linked to an anti-cancer or anti-tumor compound or agent or to a compound, agent or polypeptide which targets or otherwise directs the apoptogenic-bacteriocin to tumor or cancer cells, or to cells of a particular type or character. The apoptogenic-bacteriocin may also be covalently or non-covalently linked to any of various compounds or molecules, which will add additional functionality (e.g., an anticancer agent compound or antibody, a toxin, a distinct antigen to enable recognition by a distinct antibody, an immunomodulator) or enhance their specificity (e.g., a particular cell surface receptor-binding molecule) or stability (e.g., polymers).

The present invention also relates to a recombinant DNA molecule or cloned gene, or a degenerate variant thereof, which encodes an apoptogenic-bacteriocin; preferably a nucleic acid molecule, in particular a recombinant DNA molecule or cloned gene, that has a nucleotide sequence or is complementary to a DNA sequence capable of encoding the amino acid sequence set out in any of FIGS. 6, 7, or 8 or in SEQ ID NO: 1, SEQ ID NO: 2, SEQ ID NO:3 or SEQ ID NO:4.

The DNA sequences of the apoptogenic-bacteriocins of the present invention or portions thereof, particularly those encoding the polypeptides of any of SEQ ID NOS: 1, 2, 3, or 4, or a portion thereof, may be prepared as probes to screen for complementary or homologous sequences in other bacterial clinical isolates or other bacteria, including gram negative and gram positive bacteria. The present invention extends to probes so prepared that may be provided for screening cDNA and genomic libraries for the apoptogenic-bacteriocins. The present invention also includes the preparation of plasmids including such vectors, and the use of the DNA sequences to construct vectors expressing antisense RNA or ribozymes which would attack the mRNAs of any or all of the DNA sequences. Correspondingly, the preparation of antisense RNA and ribozymes are included herein.

In a further embodiment of the invention, the full DNA sequence of the recombinant DNA molecule or cloned gene so determined may be operatively linked to an expression control sequence which may be introduced into an appropriate host. The invention accordingly extends to unicellular hosts transformed with the cloned gene or recombinant DNA molecule comprising a DNA sequence encoding the present apoptogenic-bacteriocins(s), and more particularly, the DNA sequence capable of encoding the polypeptides of any of SEQ ID NOS: 1, 2, 3 or 4. of.

The possibilities both diagnostic and therapeutic that are raised by the existence of the apoptogenic-bacteriocins(s) derive from the fact that the apoptogenic-bacteriocins(s) are capable of killing bacterial cells and also of inducing apoptosis or cell death in particular eukaryotic cells, especially in tumor or cancer cells or cells undergoing aberrant cell growth. As suggested earlier and elaborated further on herein, the present invention contemplates pharmaceutical intervention in the growth of tumor or cancer cells and in the progression of cancer or tumors in a host, particularly in mammals, most preferably in humans. Thus, in instances where it is desired to reduce or inhibit the growth, progression, or metastasis of cancer cells, tumor cells, or cells undergoing aberrant cell growth, the apoptogenic-bacteriocins(s) of the present invention can be introduced to initiate or facilitate apoptosis or cell killing of tumor or cancer cells or otherwise block or inhibit the growth of tumors.

In a further embodiment, the apoptogenic-bacteriocin(s) of the present invention are useful in the recognition and/or killing of cells which are infected with a virus or bacteria. It is noted that there is some homology between the microcin E492 receptor and a receptor of HIV. The apoptogenic-bacteriocin(s) of the present invention may therefore be useful in recognizing, binding to and/or killing cells which are the target of or infected by viruses or bacteria, including for instance HIV. The apoptogenic-bacteriocin(s) may further be useful in recognizing, binding to and/or killing cells which are harboring bacteria or bacteria within a host by virtue of their recognition of the bacterial surface receptor(s).

The present invention thus relates to therapeutic methods based upon the activity of the apoptogenic-bacterioocin(s), its (or their) subunits, or active fragments thereof, analogs thereof, or upon agents or other drugs determined to possess the same activity. A first therapeutic method is associated with the apoptosis of tumor or cancer cells using the apoptogenic-bacteriocin(s), either individually or in mixture with each other in an amount effective to prevent, slow or inhibit the development of tumors or cancer in the host, particularly in a mammal, most particularly in a human. A further method is provided for apoptosis of tumor or cancer cells using the apoptogenic-bacteriocin(s), either individually or in mixture with each other, in an amount effective to reduce the size or growth of tumors or cancer in the host. In a further aspect, methods are provided for apoptosis of tumor or cancer cells using the apoptogenic-bacteriocin(s), in combination with an anti-tumor or anti-cancer agent or compound.

The apoptogenic-bacteriocin(s) of the present invention may be used in the treatment, prevention or modulation of any of various cancers or tumors in the host, particularly in mammals, most particularly in humans. By way of example and not limitation, cancers or tumors suitable for treatment, prevention or modulation using apoptogenis-bacteriocin(s), include, for example, cervical cancer, uterine cancer, breast cancer, ovarian cancer, prostate cancer, colon cancer, lung cancer, pancreatic cancer, liver cancer, bladder cancer, rectal cancer, brain cancer, bone cancer, cancers of unknown origin, tumor metastases, melanomas, AIDS-related tumors, Kaposi's sarcoma, Ewing's sarcoma, Wilms' tumor, adenocarcinomas, adenomas, adenopathies, Bowen's disease, carcinomas, polyps, hyperplasias, hamartoma, tissue hypertrophy, and any of various blood cell cancers, including but not limited to T cell lymphoma, B cell lymphoma, leukemia, Burkitt's lymphoma, acute lymphoblastic lymphoma (ALL), acute myelogenous leukemia (AML), Hodgkin's disease, and non-Hodgkins disease. The skilled artisan can readily assess and determine, utilizing his own knowledge and skills and based on the present description, those cancers, tumors or conditions which benefit and are modulated, treated or prevented using the apoptogenic-bacteriocin(s).

In a still further aspect, the present invention provides methods of reducing or blocking eukaryotic cell growth by administration of an apoptogenic-bacteriocin, particularly by administration of microcin E492 and/or microcin 24. In a further aspect, a method is provided for apoptosis of cells undergoing aberrant cell growth. In a particular aspect, a method is provided for treatment or prevention of conditions associated with aberrant cell growth by administration of an apoptogenic-bacteriocin, particularly by administration of microin E492 and/or microcin 24. In particular embodiments, methods are provided for treatment or prevention of conditions associated with aberrant cell growth, including but not limited to benign and malignant tumors, cancers, hyperplasias, tissue hypertrophies, psoriasis and polyps, by administration of an apoptogenic-bacteriocin, particularly by administration of microin E492 and/or microcin 24, in a particular embodiment by administration of a pharmaceutical composition comprising a polypeptide comprising the amino acid sequence of SEQ ID NO: 1, 2, 3 or 4.

The methods of the present invention includes a method for the treatment or prevention of cancer by the administration of pharmaceutical compositions comprising the apoptogenic-bacteriocin(s) of the present invention. In a particular embodiment, a method for treatment or prevention of cancer is provided comprising the administration of a pharmaceutical composition comprising microcin E492 and/or microcin 24. In a further embodiment, a method for treatment or prevention of cancer is provided comprising the administration of a pharmaceutical composition comprising a polypeptide comprising the amino acid sequence set out in any of SEQ ID NOS: 1, 2, 3 or 4. In a still further embodiment, a method for treatment or prevention of cancer is provided comprising the administration of a pharmaceutical composition comprising one or more of microcin E492 and microcin 24, in combination with an anti-tumor or anti-cancer agent or compound.

The present invention extends to the development of antibodies against the apoptogenic-bacteriocin(s) of the present invention, including in particular embodiments microcin E492 and microcin 24, including naturally raised and recombinantly prepared antibodies. Such antibodies include both polyclonal and monoclonal antibodies prepared by known genetic techniques, as well as bi-specific (chimeric) antibodies, humanized antibodies, synthetic antibodies, and antibodies including other functionalities suiting them for additional diagnostic use. Such antibodies are useful in diagnostic methods to determine the quantity, amount or activity of the apoptogenic-bacteriocin and/or to modulate the activity of the apoptogenic-bacteriocin, particularly in the case of blocking or neutralizing antibodies.

Also, antibodies including both polyclonal and monoclonal antibodies, and drugs that modulate the production or activity of the apoptogenic-bacteriocin(s) and/or their subunits may possess certain diagnostic applications and may for example, be utilized for the purpose of detecting and/or measuring conditions such as cancer, tumors, or the like. For example, the apoptogenic-bacteriocin or its subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of the apoptogenic-bacteriocin(s) of the invention may be discovered or synthesized, and may be used in diagnostic and/or therapeutic protocols.

The apoptogenic-bacteriocins and/or their antibody(ies) may, for example, be utilized in in vitro or in vivo diagnostic applications. In in vitro applications, the apoptogenic-bacteriocins and/or their antibody(ies) may be utilized, for example, on tissue, cell or biopsy samples to determine the benign or cancerous nature of the samples. In in vivo applications, the apoptogenic-bacteriocins and/or their antibody(ies) may be used, for example in in vivo imaging and diagnostic techniques, for instance by labeling them with a molecule that allows non-invasive imaging by, for example, MRI, PET, or SPECT. This use then provides a method to detect the size and position of a tumor and to follow the efficacy of therapeutic treatment. The apoptogenic-bacteriocins and/or their antibody(ies) may also be used in in vivo cancer surgery techniques, whereby, for example, labeled apoptogenic-bacteriocin(s) and/or their antibody(ies) are utilized during or after surgery to assist in determining whether cancer surgery has successfully removed the tumor and/or cancer cells. In addition, the apoptogenic-bacteriocins and/or their antibody(ies) may be used to isolate, select or eliminate cancer or tumor cells from a sample which contains cancer or tumor cells, for instance in therapeutic panning to remove cancerous or malignant cells from the blood or from bone marrow samples.

Panels of monoclonal antibodies produced against apoptogenic-bacteriocin peptides can be screened for various properties; i.e., isotype, epitope, affinity, etc. Of particular interest are monoclonal antibodies that neutralize the activity of the apoptogenic-bacteriocin or its subunits. Such monoclonals can be readily identified in apoptogenic-bacteriocin activity assays. High affinity antibodies are also useful when immunoaffinity purification of native or recombinant apoptogenic-bacteriocin is possible.

Preferably, the anti-apoptogenic-bacteriocin antibody used in the diagnostic methods of this invention is an affinity purified polyclonal antibody. More preferably, the antibody is a monoclonal antibody (mAb). In addition, it is preferable for the anti-apoptogenic-bacteriocin antibody molecules used herein be in the form of Fab, Fab′, F(ab′)₂ or F(v) portions of whole antibody molecules.

As suggested earlier, the diagnostic method of the present invention comprises examining a cellular sample or medium by means of an assay including an effective amount of an antagonist to an apoptogenic-bacteriocin protein, such as an anti-apoptogenic-bacteriocin antibody, preferably an affinity-purified polyclonal antibody, and more preferably a mAb. As previously discussed, patients capable of benefiting from this method include those suffering from cancer, a pre-cancerous lesion, or other condition resulting from aberrant cell growth.

Methods for isolating the apoptogenic-bacteriocin and inducing anti apoptogenic-bacteriocin antibodies and for determining and optimizing the ability of anti-apoptogenic-bacteriocin antibodies to assist in the examination of the target cells are all well-known in the art. The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with an apoptogenic-bacteriocin or immunogenic fragment thereof.

Splenocytes are typically fused with myeloma cells using polyethylene glycol (PEG) 6000. Fused hybrids are selected by their sensitivity to HAT. Hybridomas producing a monoclonal antibody useful in practicing this invention are identified by their ability to immunoreact with the present apoptogenic-bacteriocin and their ability to inhibit specified apoptogenic-bacteriocin activity in target cells.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8: 396 (1959)) supplemented with 4.5 gm/l glucose, 20 mm glutamine, and 20% fetal calf serum. An exemplary inbred mouse strain is the Balb/c.

The present invention further contemplates pharmaceutical compositions useful in practicing the therapeutic methods of this invention. A subject pharmaceutical composition includes, in admixture, a pharmaceutically acceptable excipient (carrier) and one or more of an apoptogenic-bacteriocin, polypeptide analog thereof or fragment thereof, as described herein as an active ingredient.

In a still further aspect, the present invention extends to a pharmaceutical composition of an apoptogenic-bacteriocin, comprising the apoptogenic-bacteriocin and a pharmaceutically acceptable carrier vehicle or diluent.

In a particular aspect, a pharmaceutical composition is provided comprising microcin E492 and a pharmaceutically acceptable carrier vehicle or diluent. In a further aspect, a pharmaceutical composition is provided comprising the polypeptide of SEQ ID NO: 1 or SEQ ID NO: 2 and a pharmaceutically acceptable carrier vehicle or diluent.

In a particular aspect, a pharmaceutical composition is provided comprising microcin 24 and a pharmaceutically acceptable carrier vehicle or diluent. In a further aspect, a pharmaceutical composition is provided comprising the polypeptide of SEQ ID NO: 4 and a pharmaceutically acceptable carrier vehicle or diluent.

The apoptogenic-bacteriocin(s) may be prepared in pharmaceutical compositions, with a suitable carrier and at a strength effective for administration by various means to a cancer patient or a patient experiencing an adverse medical condition associated with aberrant cell growth for the treatment or prevention thereof. A variety of administrative techniques may be utilized, among them parenteral techniques such as subcutaneous, intravenous and intraperitoneal injections, catheterizations and the like. Average quantities of the apoptogenic-bacteriocins or their subunits may vary and in particular should be based upon the recommendations and prescription of a qualified physician or veterinarian.

The preparation of pharmaceutical compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the pharmaceutical composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic pharmaceutical polypeptide-, analog- or active fragment-containing compositions are conventionally administered intravenously, as by injection of a unit dose, for example, and may also be administered intraperitonealy, intramuscularly, subcutaneously, orally, nasally or by other acceptable and efficacious administration routes. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of or amount of apoptogenic-bacteriocin activity desired or necessary. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The therapeutic compositions may further include an effective amount of the apoptogenic-bacteriocin, and an immunomodulator or anti-tumor or anti-cancer agent or compound, including for example, but not limited to, one or more of the following as active ingredients: interleukin(s), interferon(s), CD-40 ligand, BL22 and/or other bacterial immunotoxins, bleomycin and/or other anticancer antibiotics, anticancer antibody(ies) such as CC-49, A33 and campath-1H, and cancer chemotherapeutics such as tamoxifen and paclitaxel.

As used herein, “pg” means picogram, “ng” means nanogram, “ug” or “μg” mean microgram, “mg” means milligram, “ul” or “μl” mean microliter, “ml” means milliliter, “l” means liter.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage □, e.g., NM989, and other phage DNA, e.g., M13 and filamentous single stranded phage DNA; yeast plasmids such as the 2μ/plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptonzyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture. Contemplated in the invention are host cells, for instance and in particular embodiments bacterial host cells, expressing the apoptogenic-bacteriocin. Such apoptogenic-bacteriocin expressing cells, an in particular bacteria, may be used to infect an individual, particularly a mammal or human, and thereby cause or result in apoptosis of some specific types of eukaryotic cells, particularly tumorigenic cells. By utilizing bacteria expressing the apoptogenic-bacteriocin, particularly antibiotic sensitive bacteria, antibiotics may be utilized to control or cease the bacterial infection as desired. Such apoptogenic-bacteriocin expressing cells may thus be used in methods for treatment or prevention of conditions associated with aberrant cell growth by administration of or infection with an apoptogenic-bacteriocin expressing cell. In particular embodiments, methods are provided for treatment or prevention of conditions associated with aberrant cell growth, including but not limited to benign and malignant tumors, cancers, hyperplasias, tissue hypertrophies, psoriasis and polyps, by administration of an apoptogenic-bacteriocin expressing cell.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

It is further intended that apoptogenic-bacteriocin analogs or allelic variants may be prepared from amino acid or nucleotide sequences of the apoptogenic-bacteriocin within the scope of the present invention. Analogs, such as fragments, may be produced, for example, by pepsin digestion of apoptogenic-bacteriocin polypeptide. Other analogs, such as muteins, can be produced by standard site-directed mutagenesis of apoptogenic-bacteriocin coding sequences. In addition, due to the low molecular weight of these apoptogenic-bacteriocin(s), analogs may be generated by polypeptide synthesis, wherein amino acid substitutions, insertions or deletions can be generated. Analogs exhibiting “apoptogenic-bacteriocin activity” such as small molecules, whether functioning as promoters or inhibitors, may be identified by known in vivo and/or in vitro assays.

As mentioned above, a DNA sequence encoding an apoptogenic-bacteriocin can be prepared synthetically rather than cloned. The DNA sequence can be designed with the appropriate codons for the apoptogenic-bacteriocin amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292: 756 (1981); Nambair et al., Science, 223: 1299 (1984); Jay et al., J. Biol. Chem., 259: 6311 (1984).

Synthetic DNA sequences allow convenient construction of genes which will express apoptogenic-bacteriocin analogs or “muteins”. Alternatively, DNA encoding muteins can be made by site-directed mutagenesis of native apoptogenic-bacteriocin genes or DNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244: 182-188 (April 1989). This method may be used to create analogs with unnatural amino acids.

The present invention extends to the preparation of antisense oligonucleotides and ribozymes that may be used to interfere with the expression of the apoptogenic-bacteriocin at the translational level. This approach utilizes antisense nucleic acid and ribozymes to block translation of a specific mRNA, either by masking that mRNA with an antisense nucleic acid or cleaving it with a ribozyme. The DNA sequences described herein may thus be used to prepare antisense molecules against, and ribozymes that cleave mRNAs for the apoptogenic-bacteriocin(s) and their ligands.

Antisense nucleic acids are DNA or RNA molecules that are complementary to at least a portion of a specific mRNA molecule. (See Weintraub, 1990; Marcus-Sekura, 1988.) In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into apoptogenic-bacteriocin-producing cells. Antisense methods have been used to inhibit the expression of many genes in vitro (Marcus-Sekura, 1988; Hambor et al., 1988).

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, 1988.). Because they are sequence-specific, only mRNAs with particular sequences are inactivated.

Investigators have identified two types of ribozymes, Tetrahymena-type and “hammerhead”-type. (Hasselhoff and Gerlach, 1988) Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The invention may be better understood by reference to the following non-limiting Examples, which are provided as exemplary of the invention. The following examples are presented in order to more fully illustrate the preferred embodiments of the invention and should in no way, be construed, however, as limiting the broad scope of the invention.

### EXAMPLE 1

### Toxic Activity of Microcin E492 in Broad Range of Bacterial Strains

Microcin E492 has a broad target range toxic activity among prokaryotic cells, being active on strains of Escherichia coli, Klebsiella, Salmonella, Citrobacter, Enterobacter and Erwinia (de Lorenzo, 1984). The first step necessary for microcin action is the recognition of this bacteriocin by a receptor and translocation across the outer membrane to the inner membrane. TonB/ExbB mediate the insertion and/or translocation step because mutants in these proteins are insensitive to microcin action (Pugsley et al., 1986). TonB was demonstrated to be critical for microcin insertion into the membrane or channel formation (Lagos et al., 2001). The incapacity to find a mutant in a single outer membrane protein that completely abolished microcin recognition/translocation could mean that microcin E492 recognizes more than one receptor, increasing the recognition possibilities of different bacterial strains, and would explain the sensitivity of a variety of gram-negative bacteria to microcin E492.

Three mutants in genes encoding for iron-regulated outer membrane proteins that interact with the TonB pathway were used to assess microcin sensitivity: cir and flu, both receptor for the ferric complexes of the enterochelin-related compounds dihydroxybenzoylserine and dihydroxybenzoate, and fepA, the ferrienterochelin receptor that also participates in dihydroxybenzoylserine recognition (Hantke, 1990). To assess that the capacity of microcin E492 to kill different gram negative cells is connected with the ability to recognize more than one receptor on the surface of sensitive strains, experiments using a combination of double and triple mutants mentioned above were carried out. Table I shows that only the triple mutant (fepA, cir, flu) was completely resistant to high concentrations of microcin E492. Single and double mutant combinations were sensitive to microcin E492. This result indicates that any of the catechol receptors, FepA, Fiu or Cir are able to accomplish the microcin E492 receptor function.

TABLE I
Effect of microcin E492 on different
outer membrane protein mutants

| E. coli strain | Relevant receptor | | | % surviving cells |
|---|---|---|---|---|
| H1443* | FepA⁺ | Cir⁺ | Fiu⁺ | 0.001 |
| AB2847* | FepA⁺ | Cir⁺ | Fiu⁺ | 0.001 |
| H1758 | FepA⁺ | Cir⁺ | — | 0.001 |
| IR42 | FepA⁺ | — | Fiu⁺ | 0.0002 |
| IR20 | — | Cir⁺ | Fiu⁺ | 0.01 |
| H1728 | FepA⁺ | — | — | 0.007 |
| H1877 | — | Cir⁺ | — | 0.07 |
| H1875 | — | — | Fiu⁺ | 0.008 |
| H1876 | — | — | — | 108 |

*Wild type strain H1443 is the isogenic control for H1758, H1728, H1877, H1875 and H1876, and AB2847 is the wild type isogenic control for IR42 and IR20. Strains used are described in Hantke, 1990. To assess resistance, 5 × 10⁷ cells/ml were incubated for 15 min with a high microcin concentration, in the range of 30 μg/ml, and plated for survivals. 100% in each case corresponds to untreated cells.

Interaction of microcin E492 with TonB was studied using a genetic approach. TonB is a protein with an important periplasmic domain that is anchored to the inner membrane through a transmembrane helix located in the N-terminal region. A non-sense mutant of TonB that generated a truncated protein with the first 43 amino acids (in which the transmembrane region is located) was characterized. This mutant was sensitive to microcin E492, indicating that the transmembrane domain interacts with this bacteriocin. This result also suggests that the role of TonB is mainly at the level of microcin insertion into the membrane of the sensitive host. The periplasmic domain of TonB that interacts with the receptors Fiu, Cir or FepA was dispensable for microcin activity, suggesting that the interaction of TonB with microcin E492 does not involve a translocation step. The fact that TonB as well as the receptors Fiu, Cir and FepA are conserved in many bacterial strains is another reason for the broad target range.

### EXAMPLE 2

### The Cytotoxic Effect of Microcin E492 is Specific for Tumor-Related Human Cell Lines

Cytotoxicity of microcin E492 was assessed on several human cell lines with different morphologies and physiology: HeLa (epithelial cell line derived from human cervix carcinoma), Jurkat (T-lymphoma cell line), KG-1 (monocyte-macrophage non-tumoral derived cell line), PJ2.2.5 (a variant of Raji B-LCL cell line), Ramos (a B cell line originated from Burkitt's lymphoma) and the primary culture AMG-3 (endothelium-derived cells from human tonsils). Table II shows cell viability after incubation for 24 h in medium containing 680 AU/mL (˜14 μg/mL) of microcin E492. The two non-tumoral cell types used (KG-1 and AMG-3) were insensitive to microcin E492, Ramos presented a slight sensitivity to this bacteriocitotoxin, while HeLa, RJ2.2.5, and Jurkat cell lines were sensitive, at different degrees, to the microcin E492 toxic effect.

TABLE II
Effect of microcin E492 on different human cells types

| | Cell survival (%) |
|---|---|
| Jurkat | 4 ± 3 |
| HeLa | 56 ± 6 |
| RJ2.2.5 | 57 ± 11 |
| Ramos | 79 ± 19 |
| KG-1 | 91 ± 1 |
| AMG-3 | 99 ± 1 |

Cells were incubated for 24 h in the absence (untreated control) or presence of 680 AU/mL of microcin E492. The percentages of cell survival was determined in respect to the untreated control, and are the average of three independent measurements. Cells were stained with PI and cell death was determined by flow cytometry analysis

Microcin E492 has no hemolytic activity on blood agar, and did not present any evident toxic effect after intraperitoneal injection into mice. Hence, the cytotoxic effect observed seems to be restricted to tumoral cell lines.

Pore forming bacterial toxins, such as hemolysins and leukotoxins (RTX toxin family) exert their lethal effect by binding to cell-surface receptor identified as LFA-1, a member of the integrin family (reviewed in Lally et al., 1999). In order to determine if the cytotoxic specificity of microcin E492 correlates with the expression of LFA-1, the expression of this antigen at the cell surface of the six cell types used in this work was studied by indirect immunofluorescence, using the TS1/22.1.1 monoclonal antibody. HeLa cell line, which is sensitive to microcin E492, did not present LFA-1 at the cell surface while KG1 cell line, which is insensitive to microcin E492, expressed high cell surface levels of LFA-1. Jurkat and RJ2.2.5 cell lines expressed LFA-1, while this protein was not detected in Ramos cell line, and in the primary culture AMG-3. The lack of correlation between toxicity and expression of LFA-1 discards this protein as the microcin E492 receptor. This represents another important difference between microcin E492 and the group of bacterial toxins.

The fact that cytotoxicity of microcin is specific on particular cell types suggests that the restricted host cell specificity can be due to the binding to an unknown cell-surface receptor. The receptor does not necessarily have to be a protein. For instance, aerolysin does not recognize one specific receptor protein, but interacts with a specific post-translational modification, a glycosylphosphatidylinositol anchor (Abrami et al., 2000), and perfringolysin O, a pore-forming cytolysin, recognizes the lipid composition of the cell membrane (Shatursky et al., 1999). Microcin protein sequence was analyzed using advanced searches for domains or similarities with eukaryotic proteins that may correlate this bacteriocin with a possible receptor. However, microcin E492 did not present any recognizable domain, nor structural homologies that may connect this bacteriocin as a ligand for an eukaryotic receptor.

The role that this bacteriocin may play in pathogenicity is uncertain, because no toxic effect of microcin E492 was found using a primary culture of endothelial cells from human tonsils. In addition, microcin E492 did not produce hemolysis on blood agar, and four intraperitoneal injections of 50 μg of microcin E492 into Balb/C mice during four weeks did not have any lethal effect, indicating that the toxicity of this bacteriocin seems to be restricted to particular cell lines.

### EXAMPLE 3

### Low Doses of Microcin E492 Induce Cellular Apoptosis

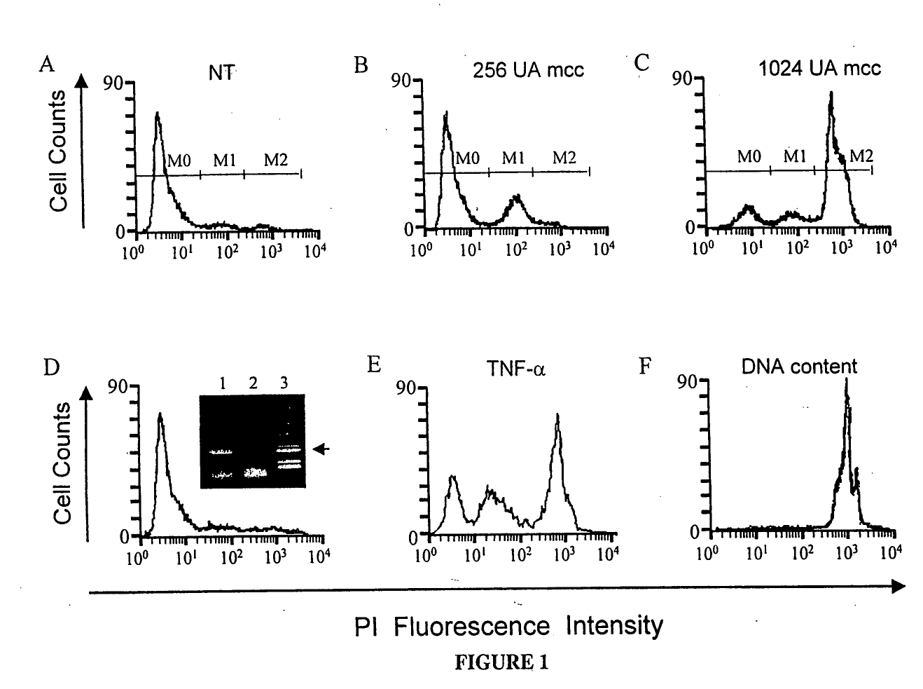

HeLa cells were chosen to characterize the cytotoxic effect of microcin E492. The propidium iodide [PI] exclusion method detects dead cells by the loss of membrane integrity. Once PI is inside the cell, it binds to DNA and its fluorescence is proportional to the DNA content. HeLa cells exposed to increasing concentrations of microcin E492 for 24 h were analyzed using this method. The controls for this experiment are depicted in FIG. 1A (M0, basal fluorescence of viable cells of the untreated control) and 1F (total DNA content of untreated cells permeabilized with methanol). After treatment with 256 AU/mL (˜5 μg/mL) of microcin E492 most dead cells showed decreased PI fluorescence (FIG. 1B) when compared with the permeabilized untreated cells stained with PI (FIG. 1F). This so-called hypodiploid part of the population (M1) would correspond to apoptotic cells with degraded DNA that bind less PI than dead cells with intact DNA. HeLa cells killed with a high dose of microcin E492 (1024 AU/mL, ˜20 μg/mL) presented mainly an intact DNA content (M2 in FIG. 1C), indicating a death by necrosis. Treatment of HeLa cells with an intermediate dose of microcin E492 (512 AU/mL, ˜10 μg/mL) produced a mixture of the two populations with different DNA content (data not shown), i.e. death by necrosis and apoptosis. Apoptosis and necrosis were also induced after treatment of HeLa cells with 1 ng/mL of TNF-α in combination with 5 μg/mL of cycloheximide (FIG. 1E). In order to discard the possibility that cytotoxicity was induced by minor contaminants that usually co-purify with microcin E492 (FIG. 1D, inset lane 1), a cytotoxic assay was carried out using a microcin preparation starting from a supernatant of the mutant strain E. coli VCS257np108. This mutant does not export microcin E492 to the extracellular space, judging by both protein electrophoresis (FIG. 1D, inset lane 2) and microcin activity measurements (Lagos et al., 2001), but produce all the other components that co-purify with microcin E492. FIG. 1D shows that this preparation had no effect on HeLa cell viability, demonstrating that the cytotoxic effect observed is associated with the presence of microcin E492.

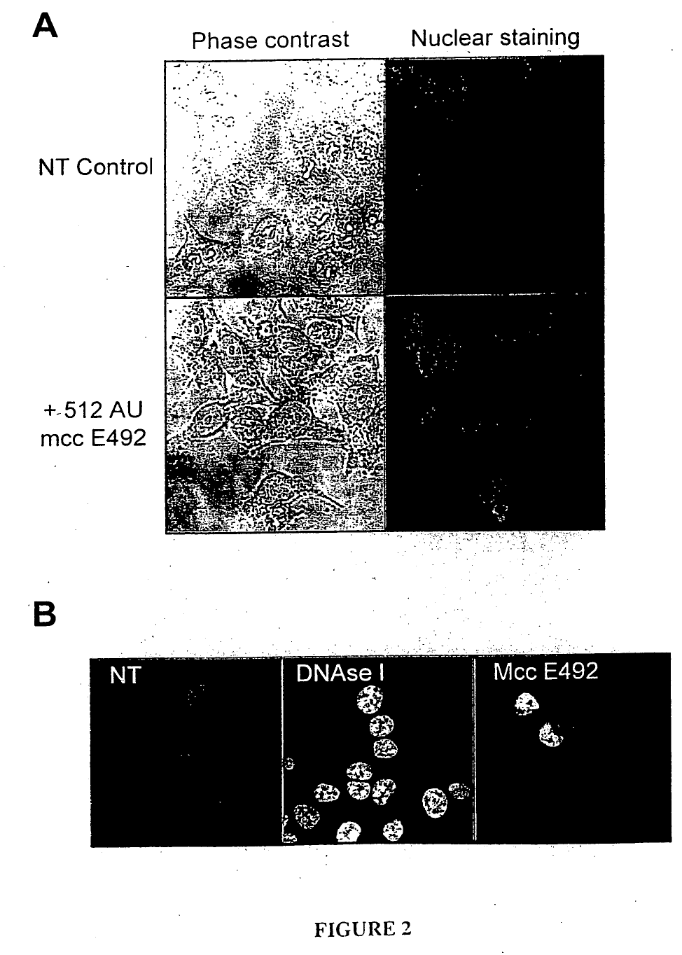

Although apoptosis and necrosis are two very different forms of cell death, the distinction between these two forms of cell death is often difficult to establish with a single technique. To confirm that microcin E492 could induce apoptosis, morphological and biochemical features typical from apoptosis were examined. Using PI staining and phase contrast microscopy, the nuclear and cell morphology of HeLa cells treated with microcin E492 were observed. After 24 h of incubation with 512 AU/mL (˜10 μg/mL) of microcin E492 cells showed nuclear changes such as chromatin condensation and nuclear fragmentation (FIG. 2A), and also the loss of epithelial morphology (rounding of the cells), with detachment from the culture plate and cell shrinkage (FIG. 2A, phase contrast). These morphological changes are typical of apoptosis. The presence of DNA fragmentation, a characteristic of apoptotic cells, was tested in situ by TdT-mediated dUTP nick-end labeling [TUNEL] analysis. FIG. 2B shows that HeLa cells treated with 512 AU/mL (˜10 μg/mL) of microcin E492 for 24 h, exhibited TUNEL-positive chromatin. As many cells were detached throughout this experiment, the proportion of TUNEL-positive cells in FIG. 2B may not accurately represent the actual percentage of the entire population of apoptotic cells.

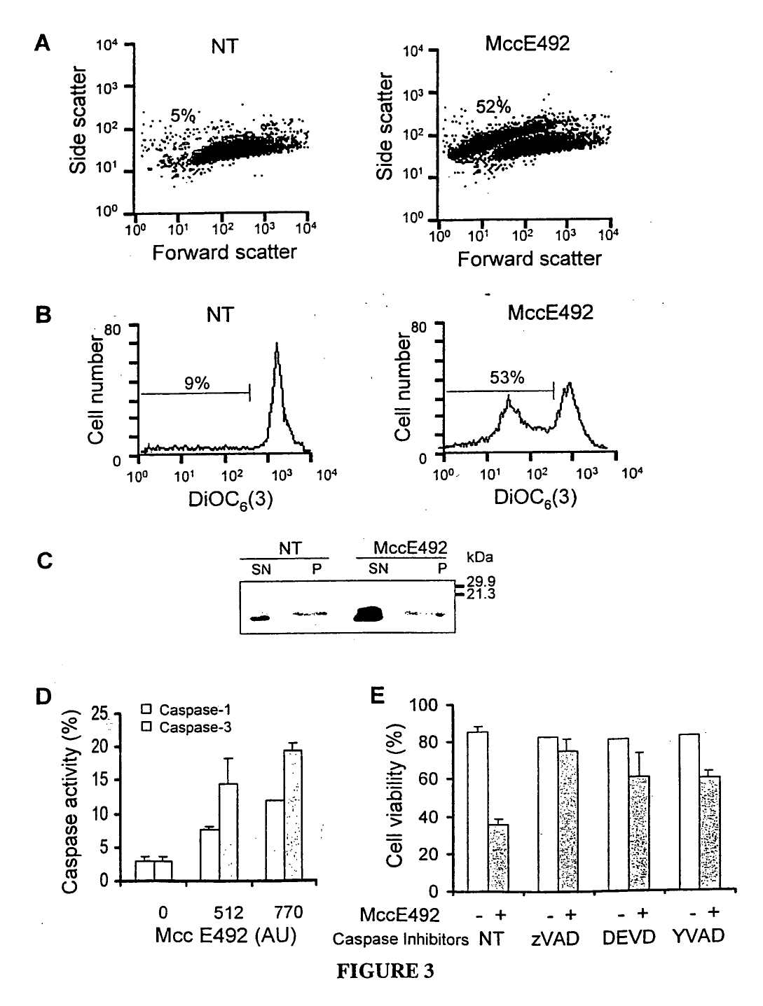

Among the most distinctive features of apoptosis are the loss of cell volume and mitochondrial dysfunction that includes the release of cytochrome c. A relationship between cell shrinkage, loss of mitochondrial membrane potential and release of cytochrome c after treatment with microcin E492 was investigated. Cell volume was assessed by light-scattering on a forward scatter versus side scatter dot plot, and DiOC₆(3), a carbocyanine dye that accumulates in active mitochondria, was used to detect changes in the mitochondrial membrane potential. FIG. 3A shows that after 10 h of treatment with 500 AU/mL (˜10 μg/mL) of microcin E492, HeLa cells presented a decrease in forward-scattered and an increase in side-scattered light, indicative of shrunken cells. Thus, HeLa cells treated with microcin E492 presented a bimodal size distribution, in which 52% of the cells had a decrease in cell volume while the non-treated control displayed only a 5% of cells with this characteristic. The loss of cell volume correlated very well with a reduction of mitochondrial membrane potential (53% of the cells), detected by a shift to lower fluorescence emission of DiOC₆(3) when compared with the untreated control (FIG. 3B). As expected, the cell population that presented a reduction in fluorescence emission corresponded to the shrunken cell population (not shown). In order to investigate if the loss of mitochondrial membrane potential was accompanied by cytochrome c release to the cytoplasm, cytochrome c content of the soluble and particulate fraction was assessed by western blot analysis. FIG. 3C shows that HeLa cells released cytochrome c to the cytoplasm (soluble fraction) after treatment with microcin E492.

Association of cytochrome c to procaspase-9 and Apaf-1 is necessary for the apoptosome formation, which in turn activates caspase-3 (Hengartner, 2000). Thus, caspase activation such as caspase-1 and caspase-3 after microcin E492 treatment was measured. FIG. 3D shows that activation of these caspases is dependent on the microcin dose used.

Activation of caspases after treatment with microcin E492 prompted us to investigate whether caspase inhibitors interfere with cell death induced by microcin. Interestingly, treatment of HeLa cells with 100 μM of zVAD-fmk, a general caspase inhibitor, completely abolished apoptosis induced by 700AU/mL (−14 μg/mL) of microcin E492 (FIG. 3E). Treatment with 100 μM Ac-DEVD-cho, a caspase-3 inhibitor, or Ac-YVAD-cho, an inhibitor of ICE proteases like caspase-1, partially counteracted the cytotoxic effect of microcin E492 on HeLa cells (FIG. 3E). These findings allow us to conclude that apoptosis induction by microcin E492 is dependent on activation of caspases.

### EXAMPLE 4

### High Doses of Microcin E492 Induce Necrosis and Apoptosis

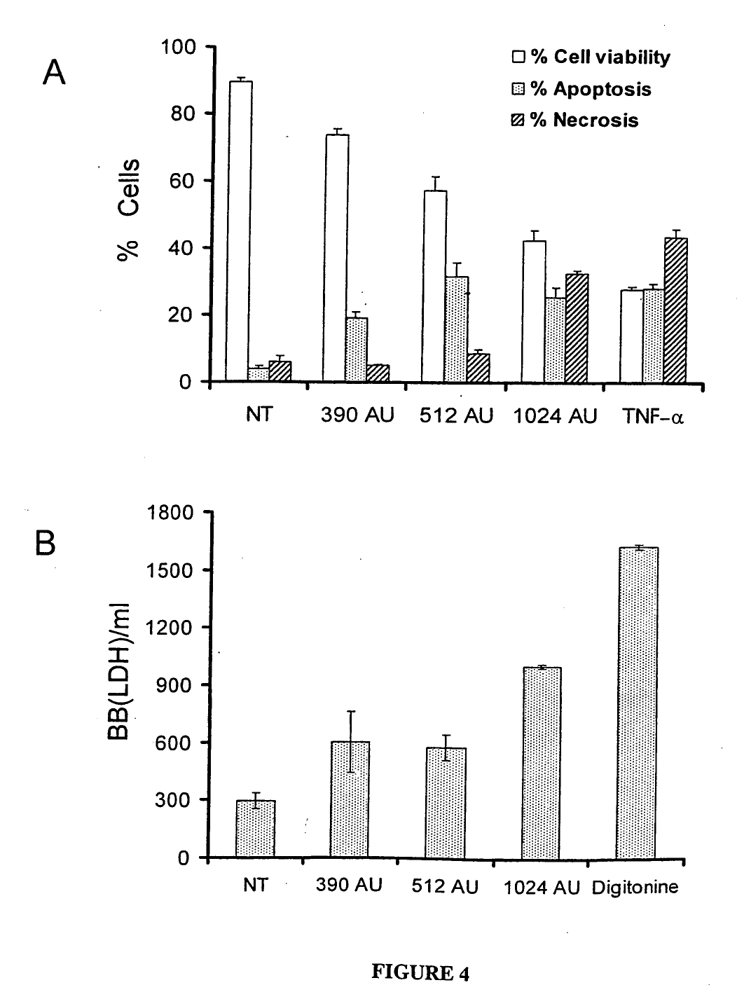

The results presented previously indicate that microcin E492 induces morphological and biochemical changes typical of apoptosis. However, many cytotoxic agents used at high doses can induce necrosis (Jonas et al., 1994; Dypbuky et al., 1994; Barros et al., 2001). FIG. 1C shows mainly the induction of cell death without DNA fragmentation after treatment with 1024 AU/mL of microcin E492, suggesting that high doses of microcin E492 could induce cell death by necrosis. In order to assess the necrosis:apoptosis ratio depending on the microcin dose used, phosphatidylserine exposure at the cell surface was measured. This change occurs in early stages of apoptosis, and can be recognized by binding of annexin V labeled with fluorescein isothiocyanate [FITC]. In early stages of apoptosis cells are not permeable to PI, and counter-staining with this fluorescent probe distinguishes the necrotic cells. HeLa cells treated with 390 and 512 AU/mL of microcin E492 for 12 h showed 19% and 32% of apoptosis respectively (FIG. 4A), while the percentage of necrotic cells was only 5 and 9%, respectively. However, HeLa cells incubated with higher doses of microcin E492 (1024 AU/mL; ˜20 μg/mL) increased the necrotic population to 32%, in contrast with the 25% of apoptosis (FIG. 4A). The apparent discrepancy in cell viability observed using the PI cytotoxic assay (FIG. 1) and annexin V may be due to the fact that the measurements of the latter were performed only after 12 h of incubation, a stage in apoptosis in which the cells are not permeable to PI, while the PI cytotoxic assay was performed after 24 h of incubation with microcin E492.

The results presented in FIG. 4A were further confirmed measuring a cell necrosis marker, the release of lactate dehydrogenase [LDH] to the culture medium. As expected, 390 and 512 AU/mL of microcin E492 induced a lower leakage of LDH when compared with HeLa cells treated with a high dose of microcin E492 (FIG. 4B).

Dose dependence associated with the induction of apoptosis or necrosis by bacterial toxins has been also described for α-toxin produced by S. aureus (Jonas et al., 1994). T lymphocytes exposed to this toxin presented DNA degradation, typical of apoptosis, after incubation with low concentrations of this toxin (<200 nM). At higher doses, cells underwent necrosis, characterized by lysis without DNA degradation.

### EXAMPLE 5

### Microcin E492 Induces Calcium Fluxes in Hela Cells

Changes in intracellular Ca²⁺ have been reported to occur at the beginning of apoptosis induced by pore-forming bacterial toxins, like α-toxin (Jonas et al., 1994), PorB (Müller et al., 1999), aerolysin (Nelson et al., 1999), and other toxins, such as verotoxin 1 (Taga et al., 1997). Microcin E492 forms ion channels in planar phospholipid bilayers (Lagos et al., 1993), and its hydropathy profile is compatible with its incorporation into the plasma membrane (Lagos et al., 1999). Therefore, we investigated the effect of microcin E492 treatment on the intracellular Ca²⁺ levels of HeLa cells. HeLa cells were loaded with the Ca²⁺-responsive fluorophore, Fluo-3, and the basal fluorescence level of the indicator-loaded cells was determined for 20 min. Basal Ca²⁺ concentration in these cells was estimated to be approximately 55 nM.

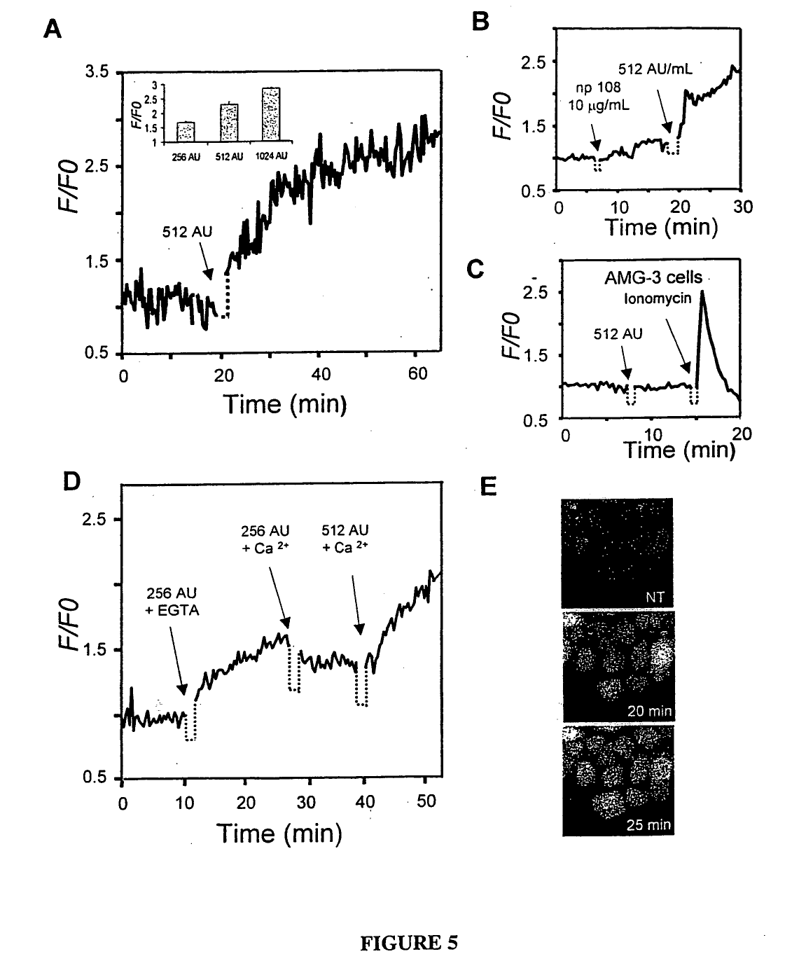

Cytosolic Ca²⁺ levels increased 2.5 fold after addition of 512 AU/mL of microcin E492 (FIG. 5A), and this effect was dose-dependent (see inset FIG. 5A). To verify that the minor contaminants of microcin did not have any effect on Ca²⁺ fluxes, a microcin preparation obtained from the supernatant of the non-producing mutant strain E. coli VCS257np108 was used (shown in inset FIG. 1D). This preparation had no significant effect on the intracellular Ca²⁺ concentration (FIG. 5B), confirming that the increase on intracellular Ca²⁺ was due to the presence of microcin E492. Treatment of the endothelial AMG-3 cells (insensitive to the cytotoxic effect of microcin E492; see Table I) with microcin did not have any effect on cytosolic Ca²⁺ levels (FIG. 5C), which supports that the cytotoxic effect is related to changes in intracellular Ca²⁺ concentrations.

A rise in cytosolic Ca²⁺ could be originated by the release of Ca²⁺ from intracellular stores, such as the endoplasmic reticulum, or by uptake from the extracellular space. FIG. 5D shows that when extracellular Ca²⁺ was chelated by the addition of 5 mM EGTA, the increase of the intracellular Ca²⁺ level was to the same extent as that observed without the chelating agent. This observation suggests that microcin E492 triggers the release of Ca²⁺ from intracellular stores, and discards the possibility that Ca²⁺ may enter through a Ca²⁺ channel, such as the case described for PorB (Müller et al., 1999). On the other hand, aerolysin promotes both Ca²⁺ influx across the plasma membrane and release of Ca²⁺ from intracellular stores (Krause et al., 1998). Increase of intracellular Ca²⁺ level due only to the release from intracellular stores have been reported for cells treated with the specific K⁺ ionophores, valinomycin, and beauvericin (Ojcius et al., 1991), and by treatment with toxins like α-toxin (Jonas et al., 1994). α-Toxin, exerts its effect by generating pores in the plasma membrane permitting equilibration of monovalents like sodium and potassium, which in turn promotes an increase of intracellular Ca²⁺ derived from intracellular storage pools (Jonas et al., 1994). In all cases described before, the rise of Ca²⁺ levels occurs during the onset of apoptosis. Ca²⁺ fluxes have been implicated in the activation of apoptosis, because depletion of Ca²⁺ stored within the endoplasmic reticulum would produce Ca²⁺ overloaded mitochondria, resulting in abnormal mitochondrial metabolism, which activates apoptosis (Berridge et al., 1998).

In order to correlate the increase of intracellular Ca²⁺ due to ion channel formation by microcin E492, changes on cell permeability by whole cell patch clamp analysis were measured, and it was found that microcin induces a rapid depolarization of the plasma membrane. It is likely that, as for α-toxin, the modified ion balance of the target cell promotes intracellular Ca²⁺ release. Alternatively, as discussed earlier for PorB (Müller et al., 2000), microcin E492 transported to the mitochondria could induce a permeability transition pore opening and cytochrome c depletion to mediate apoptosis.

When HeLa cells were treated with very high doses of microcin E492 (2048 AU/mL; ˜40 μg/mL) morphological changes typical of cell death by necrosis were observed. HeLa cells showed an increase in cell volume and membrane blebbing with a significant increase of Ca²⁺ levels (up to 4.5 fold) which preceded irreversible cell injury (cell lysis) (FIG. 5E).

### EXAMPLE 6

### Infection of Hela Cells with Microcin E492 Producing Strains Induces Cell Death

Since the cytotoxic effect of microcin E492 was assessed using different doses of this protein, we investigated the potential cytotoxic effect of microcin E492-producing strains on in vitro infections of HeLa cells. Infection of HeLa cells with Klebsiella pneumoniae RYC492 for 24 h at a multiplicity of infection (m.o.i.) of 10 resulted in 49% of HeLa cell death (Table III). A similar result was obtained when HeLa cells were incubated with 250 AU/mL (−5 μg/mL) of microcin E492 purified from K. pneumoniae RYC492 (not shown). In order to analyze the possible participation of the bacterial strain per se in the cytotoxic process, the non-producing microcin E492 bacterial strain E. coli VCS257 was transformed with pJEM15 (plasmid with the genetic determinants needed for microcin E492 production cloned in pHC79; Wilkens et al., 1997). Infection of HeLa cells with the bacterial strain E. coli VCS257pJEM15 induced 64% of cell death (Table III). HeLa cells infected with E. coli VCS257pHC79 strain presented no effect on cell viability (Table III) with respect to the untreated cells. These results strongly suggest that the cause of the cytotoxic effect of these bacterial strains is microcin E492 production.

TABLE III
Infection of HeLa cells with microcin E492
producing strains induces cell death

| | Cell survival (%) |
|---|---|
| K. pneumoniae RYC492 | 51 ± 7 |
| E. coli VCS257pJEM15 | 36 ± 4 |
| E. coli VCS257pHC79 | 90 ± 3 |
| Non-treated | 91 ± 5 |

Cells were infected at a m.o.i. of 10 with microcin E492 producing strains K. pneumoniae RYC492 and E. coli VCS257pJEM15, and the non-producing strain E. coli VCS257pHC79. After 24 h of incubation, cells were harvested and stained with PI for cell viability analysis by flow cytometry. The percentages of cell survival correspond to the cell population not stained with PI (M0 in FIG. 1), and are the average of three experiments.

Materials and Methods

The following Materials and Methods were utilized for the studies performed in Examples 1 through 6 above.

Isolation, purification, and fluorescent labeling of microcin E492. Microcin E492 was extracted from the supernatant of cultures E. coli VCS257pJEM15 or, when indicated, from K. pneumoniae RYC492, and purified according to de Lorenzo (1984). Microcin activity was estimated by the critical dilution method on a bacterial sensitive strain, and the activity was expressed in arbitrary antibiotic units (AU) (Mayr-Harting et al., 1972). Normally, 10 μg of microcin E492 corresponded to 500 AU. Labeling of microcin with fluorescamine and PAGE were carried out as described by Wilkens et al. (1997).

Cell lines and bacterial strains. The following human cell lines (ATCC) were used: HeLa, a epithelial cell line; KG1, a monocyte-macrophage cell line; RJ2.25, a variant of the Raji B-LCL (Accolla, 1983); Jurkat, a T cell derived from acute T cell leukemia, and Ramos, a B cell line originated from a Burkitt's lymphoma. The human endothelial cells from human tonsils (AMG-3) were isolated as described (Castro et al., 1996). Ramos, Jurkat, RJ2.2.5, KG1 and AMG-3 cells were maintained in RPMI 1640 medium supplemented with 10% fetal calf serum (FCS), and antibiotics. HeLa cell line were maintained in Dulbecco's modified eagle medium (DMM) with 10% FCS and antibiotics. For bacterial infection we used K. pneumoniae RYC492 described by de Lorenzo (1984), VCS257 (Stratagene) and VCS257pJEM15 (Wilkens et al., 1997).

Cytotoxicity Assay.

Cells were seeded at 2×10⁴ cells/well in 96-weel plates 24 h before microcin E492 addition, and were further incubated for 24 h with microcin E492. The cells were trypsinized and washed once with PBS, suspended in 400 μL PBS and stained with 1 μg/mL of propidium iodide (PI) for 0.1×10⁶ cells. 10,000 cells per sample were analyzed using a FACScan (Becton Diclinson, Mountain View, Calif.) with the Cell Quest software. For inhibition experiments, before adding 512 AU of microcin E492, cells were preincubated for 30 min with or without 100 μM of the caspase inhibitor Ac-DEVD-cho, Ac-YVAD-cho or zVAD-fmk. For infection experiments, HeLa cells and bacteria were centrifuged for 10 min at 500 r.p.m. and incubated for 24 h before quantification of cell viability.

Determination of morphological chances and chromatin condensation. HeLa cells were grown on 12-mm-diameter glass coverslips and treated with different doses of microcin E492. After 18 h, the medium was removed and the cells washed once with PBS. Cells were permeabilized by addition of 500 μL ice-cold methanol, incubated at −20° C. for 10 min, stained with PI (1 μl/mL), and treated with RNAse (1 μg/mL). After two washes, the slides were covered with glycerol-DABCO and viewed with a Carl Zeiss confocal microscope using an excitation wavelength of 488 nm and a 610 nm pass emission filter.

Terminal deoxynucleotidyl transferase-mediated dUTP-fluorescein Nick-End Labeling (TUNEL) assay. In situ detection of fragmented DNA was carried out by the TUNEL assay using the Apoptosis Detection System, Fluorescein (Promega, USA). HeLa cells were grown on 12-mm-diameter glass coverslips for 24 h. Microcin E492 was added, and after 24 h of incubation, cells were washed with PBS, fixed with 4% paraformaldehyde, permeabilized with 150 μM digitotin, and labeled with fluorescein-12dUTP according to the instructions of the manufacturer. All nuclei were stained with 1 μg/ml PI, and the cells treated with 1 μg/mL RNAse to eliminate non-specific labeling. TUNEL-positive and PI stained cells were visualized with a Carl Zeiss confocal microscope.

Determination of Phosphatidylserine Exposure and Lactate Dehydogenase Release.

Exposure of phosphatidylserine to the outer leaflet of the plasma membrane was determined by flow cytometry using annexin V coupled to FITC (Annexin-V-FLUOS staining kit, Boehringer Mannheim Biochemicals). Cells were seeded in 96-well plates and treated with microcin E492 as described. Then cells were collected by trypsinization, washed twice in PBS and suspended in 30 μL of binding buffer (10 mM HEPES-NaOH, 140 mM NaCl, 2.5 mM CaCl₂, pH 7.4) with annexin-V-FITC plus PI (1 μg/mL) to identify permeable (necrotic) cells. Cells were incubated for 15 min in the dark. After one wash, cells were suspended in 300 μL of binding buffer. 20,000 cells per sample were analyzed using a FACScan (Becton Dickinson, Mountain View, Calif.) with the Cell Quest software. The supernatants of each sample were analyzed for LDH activity using the Lactate Dehydrogenase kit from SIGMA Diagnostics following the manufacturer instructions (Barros et al., 2001).

Determination of intracellular calcium levels. Intracellular Ca²⁺ levels were determined as described previously (Barros et al., 2001). Briefly, cells were grown on 25 mm glass coverslips, washed in KRH-glc (10 mM Hepes, 136 mM NaCl, 4.7 mM KCl, 1.3 mM MgSO₄, 1.3 mM CaCl₂, 4.5 μL glucose), loaded for 30 min with 10 μg/mL of Fluo-3AM (Molecular Probes), and fluorescence was measured by confocal microscopy (excitation 488/emission 505 nm). 2 μg/mL of ionomycin was added at the end of each experiment to check for the correct loading of the cells, and intracellular Ca²⁺ levels were estimated as described by Kao et al. (1989), and modified in Barros et al. (2001).

Immunoflourescence assay. Human LFA-1 antigen (CD11a) was detected at the cell surface using the monoclonal antibody TS1/22.1.1 (Sánchez-Madrid et al., 1982). Subconfluents cultures were analyzed by indirect immunofluorescence assays as described (Bono et al., 1989). The analysis was performed on 10,000 cells using a FACScan (Becton Dickinson, Mountain View, Calif.) with the Cell Quest software.

Evaluation of Mitochondrial Membrane Potential. 2×10⁵ of HeLa cells in one mL were incubated with 500 AU (−10 μg) of microcin E492 for 10 h, and stained for 20 min at 37° C. with 1 μM 3,3′-dihexyloxacarbocyanine (DiOC₆(3)) (Molecular Probes) in DMEM supplemented with 10% FCS. The cells were washed once with PBS containing 2% FCS at 37° C., suspended in 400 μl of the same buffer, and analyzed by Flow Analysis Cell Sortment (FACS; Becton Dickinson, Mountain View, Calif.) using the Cell Quest program.

Cytochrome c release assay. 15×10⁶ HeLa cells with 700 AU (˜14 μg) of microcin E492 were incubated for 7 h at 37° C. in 1 mL of DMEM supplemented with 5% FCS. After this treatment, the cells were incubated for 10 min on ice in 1 mL of 10 mM HEPES (pH 7.5), 1 mM EGTA, 70 mM sucrose, 20 mM manitol, protease inhibitors and homogenized in a tissue grinder using a type-B pestle. The lysate was centrifuged at 1000 rpm for 1 min to eliminate non disrupted cells and the supernatant was centrifuged at 10000 g for 1 h. The pellet was directly resuspended in 1/10 of reducing Laemmli buffer, while 0.4 mL of soluble proteins were mixed with 0.4 mL of methanol and 0.1 mL of chloroform, spun for 5 min, and the aqueous phase was carefully removed without taking material from the interphase, mixed with 0.3 mL of methanol, spun for 5 min and the dry pellet was resuspended in Laeminli buffer. Proteins were separated under reducing conditions in 16% SDS-polyacrylamide gel electrophoresis, blotted onto a nitrocellulose membrane (BioRad), and the presence of cytochrome c was probed using a monoclonal antibody (Zyned, 33-8500). The reaction was developed utilizing a peroxidase-coupled secondary antibody, and bound antibody was detected using SuperSignal West Pico chemiluminescent substrate (Pierce).

Caspase-3/caspase-1 assay. HeLa cells were incubated with different doses of microcin for 12 h at 37° C. After harvesting with a strip, cells were washed once in ice-cold PBS and disrupted in 10 mM Tris-HCl (pH 7.4), 5 mM CHAPS, 2 mM MgCl₂, 150 mM NaCl, 5 mM EDTA, 2 mM DTT for 30 min on ice. The lysates were centrifuged for 15 min at 11500×g to remove cellular debris including the cell nucleus. The supernatant was transferred to a suitable microtiter plate and 50 μM of the colorimetric caspase-3 substrate Ac-DEVD-pNA or caspase-1 substrate Ac-YVAD-pNA was added (Calbiochem). The reaction was incubated for 1 h at 37° C. and absorbance was measured in an ELISA reader at 405 nm.

### REFERENCES

Abrami, L. Fivaz, M., van der Goot, F. G. (2000) Adventures of a pore-forming toxin at the target cell surface. Trends Microbiol. 8, 168-172.
Accolla, R. S. (1983) Human B cell variants immunoselected against a single Ia antigen subset have lost expression of several Ia antigen subsets. J. Exp. Med. 157, 1053-1058.
Baba, T., Schneewind, O. (1998) Instruments of microbial warfare: bacteriocin synthesis, toxicity and immunity. Trends Microbiol. 6, 66-71.
Barros, L. F., Stutzin, A., Calixto, A., Catalán, M., Castro, J., Hetz, C., and Hermosilla, T. (2001) Non-selective cation channels as effectors of free radical-induced rat liver cell necrosis. Hepatology 33, 114-122.
Berridge, M. J., Bootman M. D., and Lipp, P. (1998). Calcium-a life and death signal. Nature 395, 645-648.
Bono, M. R., Benech, P., Couillin, P., Alcaide-Loridan, C., Crisard, M. C, Jouin, H., Fisher, D. G., and Fellous, M. (1989) Characterization of human IFN-gamma response using somatic cell hybrids of hematopoietic and nonhematopoietic origin. Sonatic Cell Mol. Genet. 15, 513-523.
Booth, M. C., Bgie, C. P., Sahl, H.-G., Siezen, R. J., Hatter, K. L., and Gilmore, M. S. (1996) Structural analysis and proteolytic activation of Enterococcus faecalis cytolysin, a novel lantibiotic. Mol. Microbiol. 21, 1175-1184.
Castro, A., Bono, M. R., Simon, V., and Rosemblatt, M. (1996) Lymphocyte adhesion to endothelium derived from human lymphoid tissue. Eur. J. Cell. Biol. 70, 61-68.
Cramer, W. A, Heymann, J. B, Schendel, S. L, Deriy, B. N, Cohen, F. S, Elkins, P. A., and Stauffacher, C. (1995). Structure-function of the channel-forming colicins. Annu. Rev. Biophys. Biomol. Struct. 24, 611-641.
De Lorenzo, V. (1984). Isolation and characterization of microcin E492 for Klebsiella pneumoniae. Arch. Microbiol. 139, 72-75.
Dinges, M. M., Orwin, P. M., and Schlievert, P. M. (2000) Exotoxins of Staphylococcus aureus. Clin. Microbiol. Rev: 13, 16-34
Dypbukt, J. M., Ankarcrona, M., Burkitt, M., Sjoholm, A., Strom, K., Orrenius, S., and Nicotera, P. (1994). Different prooxidant levels stimulate growth, trigger apoptosis, or produce necrosis of insulin-secreting RINm5F cells. The role of intracellular polyamines. J. Biol. Chem. 269, 30553-30560.
Ennahar, S., Sashihara, T., Sonomoto, K., and Ishizaki, A. (2000) Class IIa bacteriocins: biosynthesis, structure and activity. FEMS Microbiol. Rev. 24, 85-106.
Parkas-Himsley, H., Hill, R., Rosen, B., and Lingwood, C. A. (1995) The bacterial colicin active against tumor cells in vitro and in vivo is verotoxin 1. Proc. Natl. Acad. Sci. USA. 92, 6996-7000.
Hantke, K. (1990) Dihydroxybenzoylserine- a siderophore for E. coli. FEMS Microbiol. Lett. 67, 5-8.
Hengartner, M. O. (2000) The biochemistry of apoptosis. Nature 407, 770-776.
Jack, R. W., Tagg, J. R., and Ray, B. (1995) Bacteriocins of gram-positive bacteria. Microbiol Rev. 59, 171-200.
Jacobson, M. D., Weil, M., and Raff, M. (1997) Programmed cell death in animal development. Cell 88, 347-354.
Jonas, D., Walev, I., Berger, T., Liebetrau, M., Palmer, M., and Bhakdi S. (1994) Novel path to apoptosis: Small transmembrane pores created by Staphylococcal alpha-toxin in T lymphocytes evoke internucleosomal DNA degradation. Infect. Immun. 62, 1304-1312.
Kao, J. P., Harootunian, A. T., and Tsien, R. Y. (1989) Photochemically generated cytosolic calcium pulses and their detection by Fluo-3. J. Biol. Chem. 264, 8179-8184.
Kolter, R. and Moreno, F. (1992) Genetics of ribosomally synthesized peptide antibiotics. Annu. Rev. Microbiol. 46, 141-163.
Krause, K.-H., Fivaz, M., Monod, A., and van der Goot, F. G. (1998) Aerolysin induces G-protein activation and Ca²⁺ release from intracellular stores in human granulocytes. J. Biol. Client. 273, 18122-18129.
Lagos, R., Baeza, M., Corsini, G., Hetz, C., Strahsburger, E., Castillo, J. A., Vergara, C., and Monasterio, O. (2001) Structure, organization and characterization of the gene cluster involved in the production of microcin E492, a channel-forming bacteriocin. Mol. Microbiol. 42, 229-243.
Lagos, R., Villanueva, J., and Monasterio, O. (1999) Identification and properties of the genes encoding microcin E492 and its immunity protein. J. Bacteriol. 181, 212-217.
Lagos, R., Wilkens, M., Vergara, C., Cecchi, X., and Monasterio, O. (1993) Microcin E492 forms ion channels in phospholipid bilayer membranes. FEBS Lett. 321, 145-148.
Lally, E. T., Hill, R. B., Kieba, I. R., Korostoff, J. (1999) The interaction between RTX toxins and target cells. Trends Microbiol. 7, 356-361.
Leland, P., Taguchi, J., Husain, S. R., Kreitman, R. J., Pastan, I., and Puri, R. K. (2000) Human breast carcinoma cells express type II IL-4 receptors and are sensitive to antitumor activity of a chimeric IL4-Pseudomonas exotoxin fusion protein in vitro and in vivo. Mol. Med. 6, 165-178.
Mayr-Harting, A., Hedges A. J., and Berkeley, C. W. (1972) Methods for studying bacteriocins. Methods Microbiol. 7A, 315-422.
Müller, A., Günther, D., Brinkmann, V., Hurwitz, R., Meyer T. F., and Rudel, T. (2000) Targeting of the pro-apoptotic VDAC-like porin (PorB) of Neisseria gonorrhoeae to mitochondria of infected cells. EMBO J 19, 5332-5343.
Müller, A., Günther, D., Düx, F., Naumann, M., Meyer T. F., and Rudel, T. (1999) Neisserial porin (PorB) causes rapid calcium influx in target cells and induces apoptosis by the activation of cysteine proteases. EMBO J 18, 339-352.
Nelson, K. L., Brodsky, L., and Buckley, J. T. (1999) Channel formed by subnanomolar concentrations of the toxin aerolysin trigger apoptosis of T lymphomas. Cell. Microbiol. 1, 69-74.
Ojcius, D. M., Zychlinsky, A., Zheng, L. M. and Young, J. D.-E. (1991) Ionophore-induced apoptosis: Role of DNA fragmentation and calcium fluxes. Exp Cell Res. 197, 43-49.
Podschun, R., and Ullmann, U. (1998) Klebsiella spp. as nosocomial pathogens: epidemiology, taxonomy, typing methods, and phatogenicity factors. Clin. Microbiol. Rev. 11, 589-603.
Pugsley, A. P., Moreno, F., and de Lorenzo, V. (1986) Microcin E492 insensitive mutants of Escherichia coli K12. J. Gen. Microbiol. 132, 3253-3259.
Riley, M. A. (1998) Molecular mechanisms of bacteriocin evolution. Annu. Rev. Genet. 32, 255-278.
Riley, M. A., and Gordon, D. M. (1999) The ecological role of bacteriocins in bacterial competition. Trends Microbiol. 7,129-133.
Sanchez-Madrid, F., Krensky, A. M., Ware, C. F., Robbins, E., Strominger, J. L., Burakoff, S. J., and Springer, T. A. (1982) Three distinct antigens associated with human T-lymphocyte-mediated cytolysis: LFA-1, LFA-2 and LFA-3. Proc. Natl. Acad. Sci. USA 79, 7489-7493.
Savill, J., and Fadok, V. (2000) Corpse clearance defines the meaning of cell death. Nature 407, 784-788.
Shatursky, O., Heuck, A. P., Shepard, L. A., Rossjohn, J., Parker, M. W., Johnson, A. E., and Tweten, R. K. (1999) The mechanism of membrane insertion for a cholesterol-dependent cytolysin: A novel paradigm for pore-forming toxins. Cell 99, 293-299.
Taga, S., Carlier, K., Mishal, Z., Capoulade, C., Mangeney, M., Lécluse, Y., Coulaud, D., Tétaud, C., Pritchard L. L., Tursz, T., and Wields, J. (1997) Intracellular signaling events in CD77-mediated apoptosis of Burkitt's lymphoma cells. Blood 90, 2757-2767.
Vaux, D. L., and Korsmeyer, S. J. (1999) Cell death in development. Cell 96, 245-254.
Weinrauch, Y., and Zychlinsky, A. (1999) The induction of apoptosis by bacterial pathogens. Annu. Rev. Microbiol. 53, 155-187.
Wilkens, M., Villanueva, J. E., Cofré, J., Chnaiderman, J., and Lagos, R. (1997) Cloning and expression in Escherichia coli of genetic determinants for production of and immunity to microcin E492 from Klebsiella pneumoniae. J. Bacteriol. 179, 4789-4794.

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

Various references are cited throughout this Specification, each of which is incorporated herein by reference in its entirety.

1. An isolated bacterial apoptogenic-bacteriocin capable of inducing apoptosis in eukaryotic tumor cells, cancer cells, or cells undergoing aberrant growth, wherein the apoptogenic-bacteriocin is not toxic to normal eukaryotic cells.

2. The apoptogenic-bacteriocin of claim 1 which is a pore-forming or channel forming bacterial protein of molecular weight less than 10,000.

3. The apoptogenic-bacteriocin of claim 2 which is a pore-forming or channel forming bacterial protein of molecular weight of about 2,500 to about 10,000.

4. The apoptogenic-bacteriocin of claim 1 which is microcin E492 or microcin 24.

5. An isolated bacterial apoptogenic-bacteriocin capable of inducing apoptosis in eukaryotic tumor cells, cancer cells, or cells undergoing aberrant growth, wherein the apoptogenic-bacteriocin is not toxic to normal eukaryotic cells, comprising the amino acid sequence set out in any of SEQ ID NOs:1, 2, 3 or 4 or an active portion or analog thereof.

6. An isolated derivative of the apoptogenic-bacteriocin of claim 1, wherein the apoptogenic-bacteriocin is covalently or non-covalently linked to an anti-cancer or anti-tumor compound or agent or to a compound, agent or polypeptide which targets or otherwise directs the apoptogenic-bacteriocin to tumor or cancer cells.

7. A pharmaceutical composition comprising the apoptogenic-bacteriocin of claim 1 and a pharmaceutically acceptable carrier, vehicle or diluent.

8. A pharmaceutical composition comprising the apoptogenic-bacteriocin selected from the group of microcin E492 and microcin 24 and a pharmaceutically acceptable carrier, vehicle or diluent.

9. A pharmaceutical composition comprising an apoptogenic-bacteriocin comprising an amino acid sequence selected from the group of SEQ ID NO: 1, SEQ ID NO: 2, SEQ ID NO:3 or SEQ ID NO:4 and a pharmaceutically acceptable carrier, vehicle or diluent.

10. A recombinant DNA molecule or cloned gene that has a nucleotide sequence or is complementary to a DNA sequence capable of encoding the amino acid sequence set out in any of SEQ ID NO: 1, SEQ ID NO: 2, SEQ ID NO:3 or SEQ ID NO:4, or an active portion or analog thereof.

11. A unicellular host transformed with the cloned gene or recombinant DNA molecule of claim 10.

12. A method for apoptosis of tumor cells, cancer cells or cells undergoing aberrant growth in a mammal, comprising administering to said mammal an effective amount of the apoptogenic-bacteriocin of claim 1.

13. A method for apoptosis of tumor cells, cancer cells or cells undergoing aberrant growth, comprising administering to said mammal an effective amount of the apoptogenic-bacteriocin of claim 1 in combination with an anti-tumor or anti-cancer agent or compound.

14. A method for apoptosis of tumor cells, cancer cells or cells undergoing aberrant growth in a mammal, comprising administering to said mammal an effective amount of apoptogenic-bacteriocin comprising the amino acid sequence of SEQ ID NO: 1, 2, 3 or 4, or an active portion or analog thereof.

15. A method for reducing or blocking eukaryotic cell growth in a mammal comprising administering to said mammal of an effective amount of the apoptogenic-bacteriocin of claim 1.

16. A method for reducing or blocking eukaryotic cell growth in a mammal comprising administering to said mammal an effective amount of apoptogenic-bacteriocin comprising the amino acid sequence of SEQ ID NO: 1, 2, 3 or 4, or an active portion or analog thereof.

17. A method for the treatment or prevention of cancer in a mammal by administration to said mammal of an effective amount of the apoptogenic-bacteriocin of claim 1.

18. A method for the treatment or prevention of cancer in a mammal by administration to said mammal of an effective amount of the apoptogenic-bacteriocin of claim 1 in combination with an anti-tumor or anti-cancer agent or compound.

19. A method for the treatment or prevention of cancer in a mammal by administration to said mammal of an effective amount of apoptogenic-bacteriocin comprising the amino acid sequence of SEQ ID NO: 1, 2, 3 or 4, or an active portion or analog thereof.

20. A method for apoptosis of tumor cells, cancer cells or cells undergoing aberrant growth in a mammal, comprising infecting said mammal with a bacteria, virus or other pathogen expressing an effective amount of the apoptogenic-bacteriocin of claim 1.

21. A method for apoptosis of tumor cells, cancer cells or cells undergoing aberrant growth in a mammal, comprising infecting said mammal with a bacteria, virus or other pathogen expressing an effective amount of apoptogenic-bacteriocin comprising the amino acid sequence of SEQ ID NO: 1, 2, 3 or 4, or an active portion or analog thereof.
